(19) United States
(12) Patent Application Publication
(10) Pub. No.: US 2025/0180493 A1
(43) Pub. Date: Jun. 5, 2025

(54) METHODS AND DEVICES FOR PROVIDING REAL-TIME ASPHALT DENSITY AND MOISTURE CONTENT PREDICTION WITH GROUND PENETRATING RADAR

(71) Applicant: The Board of Trustees of the University of Illinois, Urbana, IL (US)

(72) Inventors: Imad L. AL-QADI, Champaign, IL (US); Lama ABUFARES, Urbana, IL (US); Greg RENSHAW, Mahomet, IL (US); Qingqing CAO, Santa Clara, CA (US); Moshen MOTLAGH, Urbana, IL (US)

(21) Appl. No.: 18/638,851

(22) Filed: Apr. 18, 2024

Related U.S. Application Data
(60) Provisional application No. 63/460,099, filed on Apr. 18, 2023.

(51) Int. Cl. G01N 22/04; E01C 19/26; G01N 33/38; G01S 13/88; G01S 13/89

(52) U.S. Cl. CPC G01N 22/04; E01C 19/264; G01S 13/885; G01S 13/89; G01N 33/383

(57) ABSTRACT

Methods, apparatus, and storage medium for providing real-time asphalt density and/or moisture content prediction with ground penetrating radar. A method includes using raw data from a ground penetrating radar sensing an asphalt concrete at a location; processing the raw data to obtain processed data; determining an asphalt density of the asphalt concrete at the location based on the processed data according to an asphalt density prediction model; determining a compaction status of the asphalt concrete at the location based on the asphalt density and a target compaction threshold; and outputting the compaction status of the asphalt concrete at the location to an operator for determining whether to continue or stop compacting the asphalt concrete at the location. In another method, moisture content for cold recycling and existing asphalt concrete pavement is predicted in real-time from using raw from data of ground penetrating radar mounted on a vehicle.

### RELATED APPLICATION

This invention claims the benefit of U.S. Provisional Application No. 63/460,099, filed on Apr. 18, 2023, which is incorporated by reference in its entirety.

### BACKGROUND

This disclosure relates to methods and systems for providing real-time asphalt density and/or moisture content prediction with ground penetrating radar.

In the process of placing asphalt concrete pavement, a quality control measure needs to be performed to ensure the density and/or moisture level of asphalt concrete meet desired requirements. There are various problems/issues associated with the present technology to perform quality control of asphalt concrete. For examples, coring method is destructive and time-consuming; and methods associated with nuclear gauges are inaccurate, radioactive, only useful at random spots and in some cases, obstruct construction, resulting in safety concerns, and longer construction time. Some other methods in the form of a lawnmower-device that requires a worker to walk after compaction is completed and correction is impossible, to predict density from calculated dielectric properties, after construction when modifications are no longer possible.

The present disclosure provides various embodiments for providing real-time asphalt density and/or moisture content prediction with ground penetrating radar, addressing at least one of the problems/issues discussed above, achieving relatively easy, quick, accurate, and/or real-time measurements of asphalt density and/or moisture content of asphalt concrete, reducing construction time and/or cost, and/or leading to longer service lifetime of the constructed asphalt concrete.

### SUMMARY

The present disclosure relates to methods, devices, and systems for providing real-time asphalt density and/or moisture content prediction with ground penetrating radar.

The present disclosure describes a system for providing compaction status of a continuous asphalt concrete in real-time, the system includes a controller comprising a memory storing instructions, and the controller comprising a processor in communication with the memory, wherein, when the processor executes the instructions, the processor is configured to cause the controller to perform: obtaining raw data from a ground penetrating radar sensing an asphalt concrete at a location; processing the raw data to obtain processed data; determining an asphalt density of the asphalt concrete at the location based on the processed data according to an asphalt density prediction model; determining a compaction status of the asphalt concrete at the location based on the asphalt density and a target compaction threshold; and outputting the compaction status of the asphalt concrete at the location to an operator for determining whether to continue or stop compacting the asphalt concrete at the location.

In some embodiments, the system further includes a mounting structure configured to mount the controller and the ground penetrating radar antenna (e) to a compaction roller, wherein the mounting structure comprises: a plurality of steel bars configured to fix to lifting points of the compaction roller; a truss structure fixed to the plurality of bars and configured to receive the controller; and a plurality of beams, wherein, for each beam of the plurality of beams, a proximal end of the beam is configured to fix to the truss structure and a distal end of the beam is configured to mount the ground penetrating radar. The plurality of bars comprise: a telescopic bar with an adjustable length configured to fit a width of the compaction roller; and one or more steel bars with adjustable lengths configured to fix to the lifting points of the compaction roller; and the plurality of beams comprise non-metallic material.

The present disclosure describes a method for providing compaction status of a continuous asphalt concrete in real-time performed by an electronic device. The method includes obtaining raw data from a ground penetrating radar detecting an asphalt concrete at a location; processing the raw data to obtain processed data; determining an asphalt density of the asphalt concrete at the location based on the processed data according to an asphalt density prediction model; determining a compaction status of the asphalt concrete at the location based on the asphalt density and a target compaction threshold; and outputting the compaction status of the asphalt concrete at the location to an operator for determining whether to continue or stop compacting the asphalt concrete at the location.

The present disclosure describes another method for providing moisture content of a continuous asphalt concrete in real-time performed by an electronic device. The method includes obtaining raw data from a ground penetrating radar detecting an asphalt concrete at a location; processing the raw data to obtain processed data; determining a moisture content of the asphalt concrete at the location based on the processed data according to a moisture content prediction model; determining a moisture status of the asphalt concrete at the location based on the moisture content and a target moisture threshold; and outputting the moisture status of the asphalt concrete at the location to an operator for determining whether to close or open the asphalt concrete for traffic.

The present disclosure describes a non-transitory computer readable storage medium storing computer readable instructions. The computer readable instructions, when executed by a processor, are configured to cause the processor to perform any of the above methods.

The present disclosure also describes an apparatus including electric circuitry configured to implement any of the above methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The system, device, product, and/or method described below may be better understood with reference to the following drawings and description of non-limiting and non-exhaustive embodiments. The components in the drawings are not necessarily to scale. Emphasis instead is placed upon illustrating the principles of the present disclosure.

FIG. 1A shows a schematic diagram of an exemplary embodiment in the present disclosure.

FIG. 1B shows an exemplary embodiment in the present disclosure.

FIG. 2A shows computer systems that may be used to implement electronic components in the present disclosure.

FIG. 2B shows a schematic diagram of an electronic device in the present disclosure.

FIG. 2C shows a schematic diagram of another electronic device in the present disclosure.

FIG. 3 shows a flow diagram of an embodiment of a method in the present disclosure.

FIG. 4 shows a flow diagram of another embodiment of a method in the present disclosure.

FIG. 5 shows a global position system unit attached to the top of the controls box in a thin waterproof container to allow signal transmission.

FIG. 6 shows C-clamps connecting the mount to the roller's upper and lower lifting points.

FIG. 7 shows an exemplary antenna height and fiberglass beams length.

FIG. 8 shows a telescopic steel bar extended to fit another roller.

FIG. 9 shows a truss structure and front view exemplary dimensions in inches.

FIG. 10 shows one or more U-clamps connecting the fiberglass beams to the truss structure.

FIG. 11 shows the GPR survey configuration and received signal: (a) GPR field tests and (b) reflected signal from a trace.

FIG. 12 shows the calculated G_{mb} and air voids comparisons between signals with a different instability index: (a) G_{mb} and (b) air voids.

FIG. 13 shows the relationship between air- and pavement-coupling amplitudes.

FIG. 14 shows the time-domain signal and its PSD before and after bandpass filtering: (a) time-domain signal and (b) PSD.

FIG. 15 shows the transmitted signal and ideal signal: (a) original signal (pulse in the red square is the transmitted signal) and (b) Ricker wavelet (normalized).

FIG. 16 shows an illustration of an adaptive filter.

FIG. 17 shows the loss progression.

FIG. 18 shows the comparison among two methods and the original signal: (a) filtration on the transmitted pulse and (b) filtration on the entire scan.

FIG. 19 shows the simulation model and results: (a) Simulation model configurations and (b) relationship between peak amplitude and antenna height.

FIG. 20 shows the Relationship among dielectric constant error, h_{c} to hₚ ratio, and uncorrected dielectric constant.

FIG. 21 shows the simulation results: (a) B-scan of the simulation and (b) back-calculated dielectric constant.

FIG. 22 shows the laboratory tests and results: (a) laboratory tests set-up and (b) relationship between the reflected amplitude and height from the lab tests.

FIG. 23 shows the test settings and results from the bumper jumping test: (a) test configuration and (b) reflected amplitude vs. antenna height.

FIG. 24 shows the application of the height-correction method on dynamic tests.

FIG. 25 shows the GPR static survey on the test locations.

FIG. 26 shows the approach to collect loose samples.

FIG. 27 shows a picture of filling and compacting sand cone and sampling holes.

FIG. 28 shows the sand cone test on a CIR surface.

FIG. 29 shows the Field data over time from IL-61 project: (a) moisture content trend for locations 1-4, (b) moisture content trend for locations 5-7, (c) dielectric constant trend for locations 1-4, and (d) dielectric constant trend for locations 5-7, (e) dielectric constant vs moisture content for all collected data from IL-61 project.

FIG. 30 shows typical testing of a laboratory slab.

FIG. 31 shows the TesSecond slab dielectric constant vs. volume of water trend.

FIG. 32 shows a second slab dielectric constant vs. moisture content trend for TWTT data.

FIG. 33 shows the ACA model moisture content prediction performance.

FIG. 34 shows the ACA model's sensitivity to input density.

FIG. 35 shows (a) Diagram for the heterogeneous asphalt concrete (AC) model in gprMax and (b) effect of aggregate dielectric constant on simulated ground-penetrating radar signal.

FIG. 36 shows the effect of aggregate dielectric constant on (a) predicted bulk density (Gmb) and (b) predicted air voids (AV).

FIG. 37 shows (a) Stacks of aggregates in a conventional oven, (b) dried aggregates' buckets sealed with plastic wrap, (c) built wood frame, (d) copper plate inserted at the bottom of the wood frame and covered with a thin plastic sheet, (e) weighing of aggregates' bucket before adding to the wood frame, and (f) surface leveling using a metallic rod.

FIG. 38 shows the ground-penetrating radar test setup for aggregates.

FIG. 39 shows an example ground-penetrating radar trace from aggregate slab.

FIG. 40 shows effect of chemical elements on aggregate dielectric constant.

FIG. 41 shows Asphalt slab preparation and testing: (a) sampling from Sandeno East, IL, (b) reheating and remixing, (c) hand tamping, and (d) ground-penetrating radar test setup.

FIG. 42 shows images of a parking lot at Joliet, IL: (a) construction and (b) asphalt concrete cross-section.

FIG. 43 shows an image of the mount on a field project.

### DETAILED DESCRIPTION OF THE DISCLOSURE

The disclosed systems, devices, and methods will now be described in detail hereinafter with reference to the accompanied drawings that form a part of the present application and show, by way of illustration, examples of specific embodiments. The described systems and methods may, however, be embodied in a variety of different forms and, therefore, the claimed subject matter covered by this disclosure is intended to be construed as not being limited to any of the embodiments. This disclosure may be embodied as methods, devices, components, or systems. Accordingly, embodiments of the disclosed system and methods may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” or “in other embodiments” as used herein does not necessarily refer to a different embodiment. It is intended, for example, that claimed subject matter may include combinations of exemplary embodiments in whole or in part. Moreover, the phrase “in one implementation”, “in another implementation”, “in some implementations”, or “in some other implementations” as used herein does not necessarily refer to the same implementation(s) or different implementation(s). It is intended, for example, that claimed subject matter may include combinations of the disclosed features from the implementations in whole or in part.

In general, terminology may be understood at least in part from usage in context. For example, terms, such as “and”, “or”, or “and/or,” as used herein may include a variety of meanings that may depend at least in part upon the context in which such terms are used. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure relates to methods for providing real-time asphalt density and/or moisture content prediction with ground penetrating radar (GPR).

In the process of placing asphalt concrete pavement, the laboratory-designed mix must be achieved. Mix volumetrics (density, air voids, percent binder . . . etc.) may determine the structural capacity and service life of the pavement, and thus, predicting the density of the asphalt concrete pavement can help guide workers to provide and achieve the target density. Ground penetrating radar (GPR) may be used because it is nondestructive, highly accurate, quick, safe, and covers a large area. In some implementations, further construction on or motor vehicle use of the recycled asphalt concrete requires a maximum threshold of moisture content, for example, during a pavement rehabilitation process.

In the process of placing asphalt concrete pavement, a quality control measure needs to be performed to ensure the density and/or moisture level of asphalt concrete meet desired requirements. There are various problems/issues associated with the present technology to perform quality control of asphalt concrete, only useful at random locations and in some cases, obstruct construction, resulting in safety concerns, longer construction time. Some other methods in the form of a lawnmower-device that requires a worker to walk after compaction is completed and correction is impossible, to predict density from calculated dielectric properties, after construction when modifications are no longer possible.

The present disclosure provides various embodiments for providing real-time asphalt density and/or moisture content prediction with ground penetrating radar, addressing at least one of the problems/issues discussed above, achieving relatively easy, quick, accurate, and/or real-time measurements of asphalt density and/or moisture content of asphalt concrete, reducing construction time and/or cost, and/or leading to longer service lifetime of the constructed asphalt concrete.

In the present disclosure, a tested and refined model for density and/or moisture content prediction may be used. In some implementation, there may be one single model with various parameters. In some implementations, there may be different models, for example, one asphalt density prediction model and another moisture content prediction model.

In some implementations, the model(s), for example, Al-Qadi Cao Abufares (ACA) model, may be based on the dielectric mixing theory to predict density and moisture content of non-dry asphalt materials. This model is theoretical and requires no calibration. The model may be successfully performed on several different asphalt materials including hot mix asphalt, cold in place recycled asphalt, cold central plant recycling materials, stone mastic asphalt, warm mix asphalt, and other asphaltic materials. In some implementations, a software may be developed based on the described methods and includes the model and other previous works/algorithms to provide real-time density and/or moisture content predictions on field from raw GPR data.

In some implementations, the model(s) may be used for asphalt density and moisture content prediction, which are important for quality control and quality assurance during the construction of new pavements or evaluating existing pavements for density and moisture presence, which can lead to distresses and costly rehabilitations. The method/software can be used on field during construction of different asphaltic roads to monitor density (specifically important for new constructed asphalt concrete) or monitor moisture content (specifically important for cold recycled treatments). This monitoring is done in real-time and can help in decision making for opening roads for traffic and/or placing overlays, leading to better management and decreased rehabilitation costs.

In some implementations, other asphalt concrete density models may be used, which may require lab fabricated samples for calibration and obtaining an empirical GPR-density relationship, and may require previous knowledge of the mix design and lab work for creating the samples and testing them.

The present disclosure describes various embodiments including a mounting structure for ground penetrating radar (GPR) on various paving material roller compactor types, so as to achieve the goal of monitoring the field density of paving materials, including asphaltic materials, in real-time using an associated tool for data processing. In some implementations, the mounting structure (or referred as “mount”) is rigid, stable, and non-obstructive for the compactor operator. The mount provides no interference to GPR signal from the roller body. The mount is designed to be adjustable to fit any compaction roller, including various types, sizes, and/or models.

The mount described in the present disclosure addresses at least one of the problem/issues associated with other implementations, for example, a mounted GPR antennas on walking carts wherein users walk behind a cart, which may require calibration by lab-fabricated samples/cores to build mix-specific regression models, and/or may not allow in-situ density corrections. The mount described in the present disclosure limits user interaction (e.g., requires little or no interaction) with the mounted GPR to ensure GPR operator safety and real-time paving material density prediction. The real-time measurement and prediction using the associated tool allow the operator to apply density correction in real-time and make decisions on field.

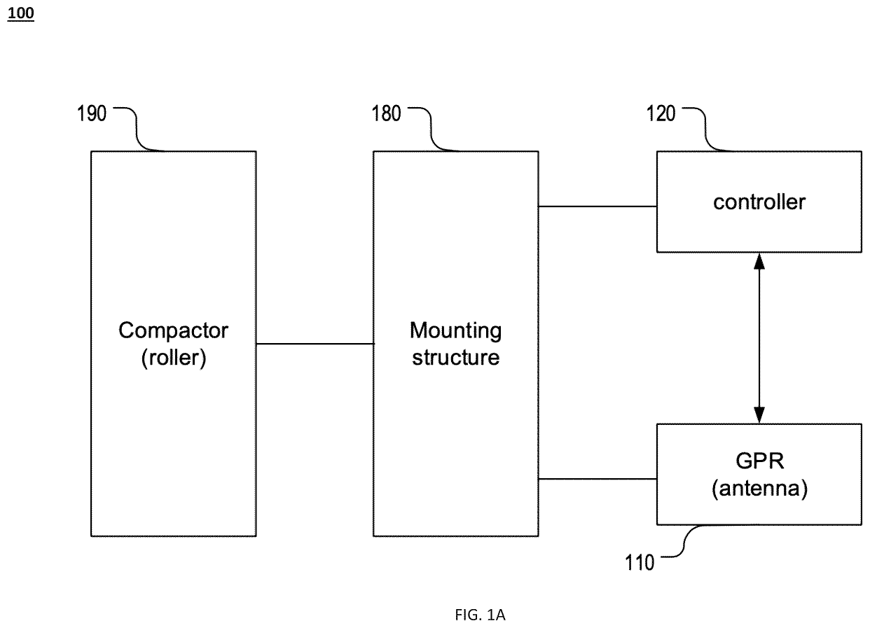

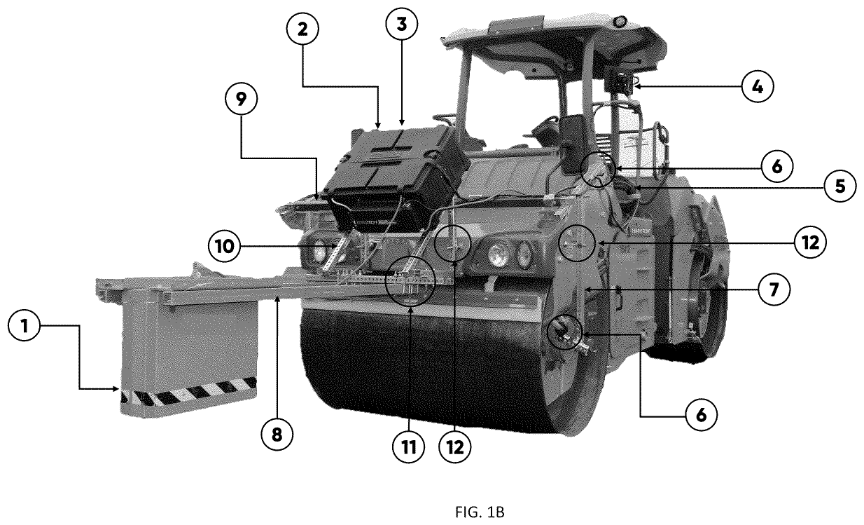

FIG. 1A shows an exemplary system 100 for providing real-time asphalt density and/or moisture content prediction with ground penetrating radar (GPR). The system 100 may include a portion or all of the following: a mount 180 attached to a compactor (roller) 190, a GPR antenna (sensor) 110 fixed to the mount, and/or a controller 120 communicating with the antenna and fixed to the mount.

FIG. 1B shows a view of another exemplary system 100, which may include a portion or all of the following: 1. GPR antenna (or any commercial antenna) connected to the system using coaxial cables; 2. Waterproof controls box which includes: GPR data collection unit, mini-computer for data analysis, AC-DC convertor, and all cable connections; 3. global position device/system (e.g., GPS); 4. Digital monitor for operator's use; 5. Power cables connecting the controls box to the roller's battery; 6. Mount to roller's lifting point connection through special bolts and clamps; 7. Adjustable steel bars; 8. Composite beams to hold the antenna at a specific distance from the roller; 9. Telescopic steel bar to adjust bar length across roller width; 10. Truss structural design for carrying the controls box and antenna weights; 11. Bolted clamps for connecting antenna beams to the truss; and/or 12. Stainless steel adjustable leveling to hold the mount away from the roller's body. The various components of the system are discussed in detail in later paragraphs.

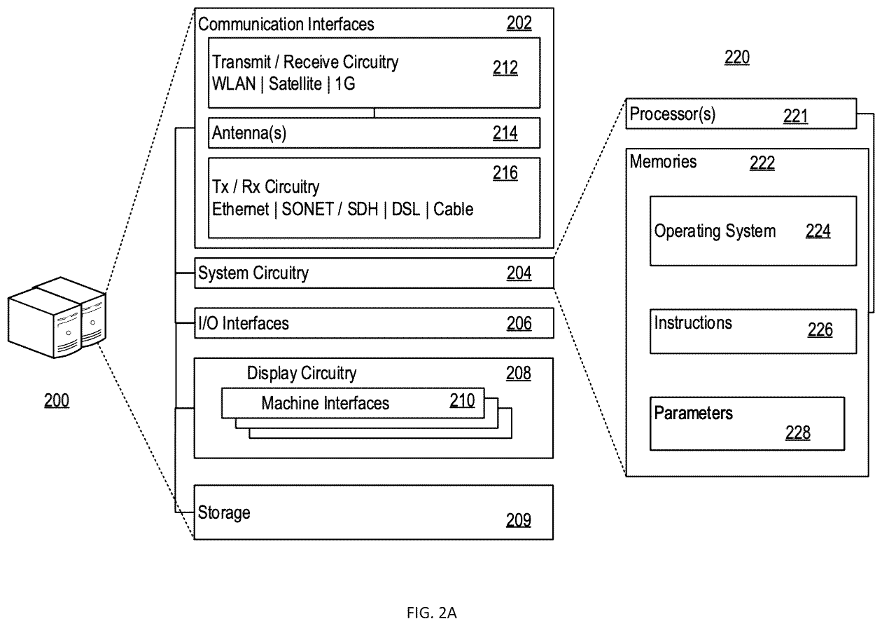

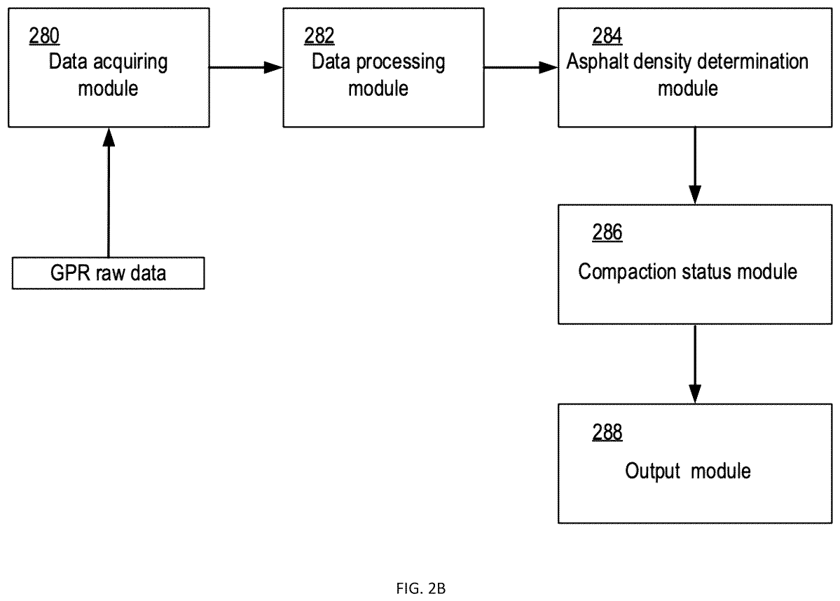

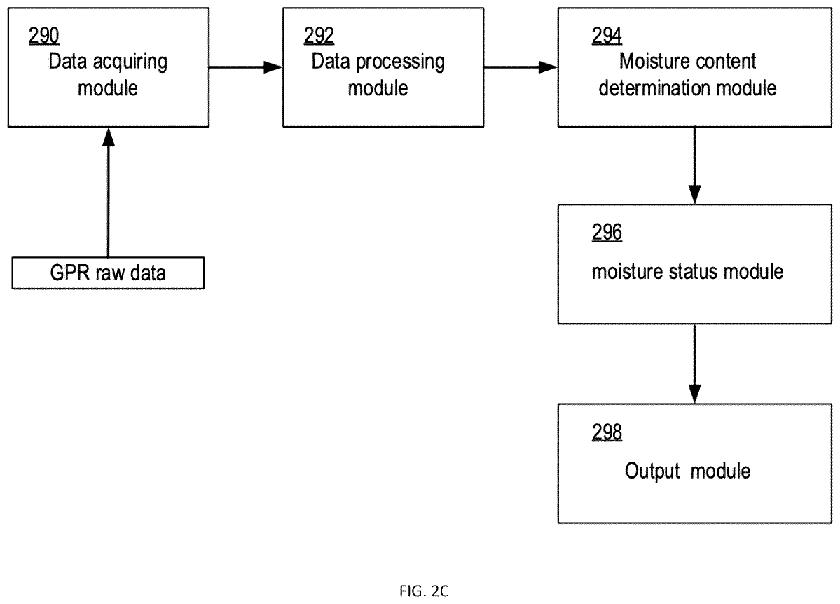

FIG. 2A shows an exemplary electronic device (e.g., the controller in FIG. 1A) for performing certain functions to provide real-time asphalt density and/or moisture content prediction with ground penetrating radar. The electronic device may comprise a computer system 200 for implementing one or more steps in various embodiments of the present disclosure. The computer system 200 may include a portion or all of the following: communication interfaces 202, system circuitry 204, input/output (I/O) interfaces 206, storage 209, and/or display circuitry 208 that generates machine interfaces 210 locally or for remote display, e.g., in a web browser running on a local or remote device. For one example, the computer system 200 may communicate with one or more instrument (e.g., the GPR, the GPS, audio speaker and/or video display, etc.).

The machine interfaces 210 and the I/O interfaces 206 may include GUIs, touch sensitive displays, voice or facial recognition inputs, buttons, switches, speakers and other user interface elements. Additional examples of the I/O interfaces 206 include microphones, video and still image cameras, headset and microphone input/output jacks, Universal Serial Bus (USB) connectors, general purpose digital interface (GPIB), peripheral component interconnect (PCI), PCI extensions for instrumentation (PXI), memory card slots, and other types of inputs. The I/O interfaces 206 may further include magnetic or optical media interfaces (e.g., a CDROM or DVD drive), serial and parallel bus interfaces, and keyboard and mouse interfaces.

The communication interfaces 202 may include wireless transmitters and receivers (“transceivers”) 212 and any antennas 214 used by the transmitting and receiving circuitry of the transceivers 212. The transceivers 212 and antennas 214 may support Wi-Fi network communications, for instance, under any version of IEEE 802.11, e.g., 802.11n or 802.11ac. The communication interfaces 202 may also include wireline transceivers 216. The wireline transceivers 216 may provide physical layer interfaces for any of a wide range of communication protocols, such as any type of Ethernet, data over cable service interface specification (DOCSIS), digital subscriber line (DSL), Synchronous Optical Network (SONET), or other protocol.

The storage 209 may be used to store various initial, intermediate, or final data or model for implementing various embodiments described in the present disclosure. These data corpus may alternatively be stored in a database. In one implementation, the storage 209 of the computer system 200 may be integral with a database. The storage 209 may be centralized or distributed, and may be local or remote to the computer system 200. For example, the storage 209 may be hosted remotely by a cloud computing service provider.

The system circuitry 204 may include hardware, software, firmware, or other circuitry in any combination. The system circuitry 204 may be implemented, for example, with one or more systems on a chip (SoC), application specific integrated circuits (ASIC), microprocessors, discrete analog and digital circuits, and other circuitry. For example, at least some of the system circuitry 204 may be implemented as processing circuitry 220. The processing circuitry 220 may include one or more processors 221 and memories 222. The memories 222 stores, for example, control instructions 226, parameters 228, and/or an operating system 224. The control instructions 226, for example may include instructions for implementing various components of the embodiment for determining at least one reaction condition. In one implementation, the instruction processors 221 execute the control instructions 226 and the operating system 224 to carry out any desired functionality related to the embodiment in the present disclosure.

FIGS. 2B and 2C describe two exemplary embodiments for providing real-time asphalt density and/or moisture content prediction with ground penetrating radar (GPR). A electronic device may include a portion or all of the following modules: a data acquiring module (280 and/or 290) that obtaining raw data from a GPR sensing an asphalt concrete at a location; a data processing module (282 and/or 292) that processes the raw data to obtain processed data; an asphalt density determination module (284) that determines an asphalt density of the asphalt concrete at the location based on the processed data according to an asphalt density prediction model; a compaction status module (286) that determines a compaction status of the asphalt concrete at the location based on the asphalt density and a target compaction threshold; an output module (288) that outputs the compaction status of the asphalt concrete at the location to an operator for determining whether to continue or stop compacting the asphalt concrete at the location; an moisture content determination module (294) that determines a moisture content of the asphalt concrete at the location based on the processed data according to a moisture content prediction model; a moisture status module (296) that determines a moisture status of the asphalt concrete at the location based on the moisture content and a target moisture threshold; and/or an output module (298) that outputs the moisture status of the asphalt concrete at the location to an operator for determining whether to close or open the asphalt concrete for traffic.

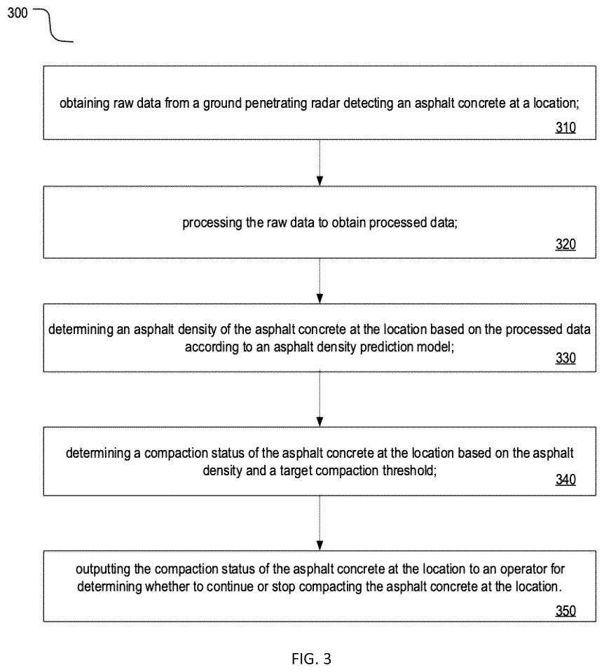

Referring to FIG. 3, the present disclosure describes various embodiments for a method 300 for providing compaction status of a continuous asphalt concrete in real-time. The method may be performed by the electronic device in FIG. 2A and may correspond to the schematic diagram in FIG. 2B. The method may include a portion or all of the following steps: step 310, obtaining raw data from a ground penetrating radar sensing an asphalt concrete at a location; step 320, processing the raw data to obtain processed data; step 330, determining an asphalt density of the asphalt concrete at the location based on the processed data according to an asphalt density prediction model; step 340, determining a compaction status of the asphalt concrete at the location based on the asphalt density and a target compaction threshold; and/or step 350, outputting the compaction status of the asphalt concrete at the location to an operator for determining whether to continue or stop compacting the asphalt concrete at the location using simple visualization techniques like GPS based heatmaps.

In some implementations, alternatively, optionally, and/or additional to any implementation/embodiment or any combination of implementations/embodiments in the present disclosure, while the ground penetrating radar is moving along the asphalt concrete, repeating steps from step 310, obtaining raw data, to step 350, outputting the compaction status, with respect to the asphalt concrete continuously at a plurality of locations.

In some implementations, alternatively, optionally, and/or additional to any implementation/embodiment or any combination of implementations/embodiments in the present disclosure, the determining the compaction status of the asphalt concrete at the location based on the asphalt density and the target compaction threshold comprises: in response to the asphalt density being smaller than the target compaction threshold, determining the compaction status of the asphalt concrete at the location as under-compaction with a color code corresponding to the asphalt density; in response to the asphalt density being equal to the target compaction threshold, determining the compaction status of the asphalt concrete at the location as at-target-compaction with a color code corresponding to the asphalt density; and/or in response to the asphalt density being larger than the target compaction threshold, determining the compaction status of the asphalt concrete at the location as over-compaction with a color code corresponding to the asphalt density.

In some implementations, alternatively, optionally, and/or additional to any implementation/embodiment or any combination of implementations/embodiments in the present disclosure, the target compaction threshold corresponds to 93% compaction.

In some implementations, alternatively, optionally, and/or additional to any implementation/embodiment or any combination of implementations/embodiments in the present disclosure, outputting the compaction status of the asphalt concrete at the location to an operator for determining whether to continue or stop compacting the asphalt concrete at the location comprises: generating a heatmap based on color codes for the asphalt concrete at a plurality of locations; and/or outputting the heatmap to an operator for determining whether to continue or stop compacting the asphalt concrete at the location.

In some implementations, for each location on the asphalt concrete pavement, the color code may be a color ranging from “red” to “green” corresponding to the asphalt density value with respect to the target compaction threshold, so that a heatmap is generated for a plurality of locations on the asphalt concrete pavement. when the operator (e.g., the driver of a compactor roller) sees the heatmap with color coding, the operator knows which areas need further compaction, and which areas have been compacted to the target density. For non-limiting example, the color is “red” when the asphalt density value is below the target compaction threshold, “yellow” when the asphalt density value is about the same as the target compaction threshold, and/or “green” when the asphalt density value is above the target compaction threshold.

In some implementations, alternatively, optionally, and/or additional to any implementation/embodiment or any combination of implementations/embodiments in the present disclosure, the compaction status of the asphalt concrete at the location being under-compaction is configured for the operator to continue compacting the asphalt concrete that the location; and/or the compaction status of the asphalt concrete at the location being over-compaction is configured for the operator to stop compacting the asphalt concrete that the location.

In some implementations, alternatively, optionally, and/or additional to any implementation/embodiment or any combination of implementations/embodiments in the present disclosure, the method further includes obtaining geo-location data of the asphalt concrete at the location from a global positioning system; and/or associating the geo-location data and the asphalt density of the asphalt concrete at the location.

In some implementations, alternatively, optionally, and/or additional to any implementation/embodiment or any combination of implementations/embodiments in the present disclosure, the processing the raw data comprises at least one of the following: pre-processing the raw data, performing signal instability correction; performing height correction; performing thin layer processing; performing surface moisture removal; and/or performing wavelet smoothing. For example, performing height correction may eliminate/reduce effects of GPR's mechanical vibration during compactor movement. For another example, performing surface moisture removal may eliminate/reduce effects of moisture on surface of the pavement during constructing asphalt pavement.

In some implementations, alternatively, optionally, and/or additional to any implementation/embodiment or any combination of implementations/embodiments in the present disclosure, the ground penetrating radar is configured to mount to a compaction roller by a mounting structure, wherein the mounting structure comprises: a plurality of bars configured to fix to lifting points of the compaction roller; a truss structure fixed to the plurality of bars and configured to receive the electronic device; and/or a plurality of beams, wherein, for each beam of the plurality of beams, a proximal end of the beam is configured to fix to the truss structure and a distal end of the beam is configured to mount the ground penetrating radar antenna.

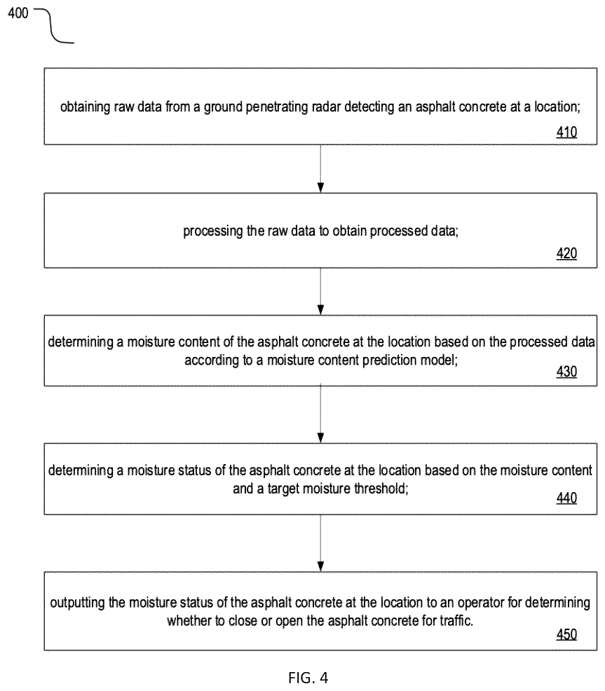

Referring to FIG. 4, the present disclosure describes various embodiments for another method 400 for providing moisture status of a continuous asphalt concrete in real-time. The method may be performed by the electronic device in FIG. 2A and may correspond to the schematic diagram in FIG. 2C. The method may include a portion or all of the following steps: step 410, obtaining raw data from a ground penetrating radar detecting an asphalt concrete at a location; step 420, processing the raw data to obtain processed data; step 430, determining a moisture content of the asphalt concrete at the location based on the processed data according to a moisture content prediction model; step 440, determining a moisture status of the asphalt concrete at the location based on the moisture content and a target moisture threshold; and/or step 450, outputting the moisture status of the asphalt concrete at the location to an operator for determining whether to close or open the asphalt concrete for traffic.

In some implementations, alternatively, optionally, and/or additional to any implementation/embodiment or any combination of implementations/embodiments in the present disclosure, while the ground penetrating radar is moving along the asphalt concrete, repeating from step 410, obtaining raw data, to step 450, outputting the moisture status, with respect to the asphalt concrete continuously at a plurality of locations.

In some implementations, alternatively, optionally, and/or additional to any implementation/embodiment or any combination of implementations/embodiments in the present disclosure, the method further includes calibrating the moisture content prediction model with a sample with a known moisture content.

In some implementations, alternatively, optionally, and/or additional to any implementation/embodiment or any combination of implementations/embodiments in the present disclosure, while the determining the moisture status of the asphalt concrete at the location based on the moisture content and the target moisture threshold comprises: in response to the moisture content being smaller than or equal to the target moisture threshold, determining the moisture status of the asphalt concrete at the location as dry; and/or in response to the moisture content being larger than the target moisture threshold, determining the moisture status of the asphalt concrete at the location as wet.

In some implementations, alternatively, optionally, and/or additional to any implementation/embodiment or any combination of implementations/embodiments in the present disclosure, the target moisture content threshold comprises 2% by weight of the asphalt mix.

In some implementations, alternatively, optionally, and/or additional to any implementation/embodiment or any combination of implementations/embodiments in the present disclosure, the moisture status of the asphalt concrete at the location being dry is configured for the operator to open the asphalt concrete for traffic; and/or the moisture status of the asphalt concrete at the location being wet is configured for the operator to close the asphalt concrete from traffic.

In some implementations, alternatively, optionally, and/or additional to any implementation/embodiment or any combination of implementations/embodiments in the present disclosure, the ground penetrating radar is configured to mount to a compaction roller by a mounting structure, wherein the mounting structure comprises: a plurality of bars configured to fix to lifting points of the compaction roller; a truss structure fixed to the plurality of bars and configured to receive the electronic device; and a plurality of beams, wherein, for each beam of the plurality of beams, a proximal end of the beam is configured to fix to the truss structure and a distal end of the beam is configured to mount the ground penetrating radar.

In various embodiments/implementations, the density/moisture content prediction model(s) may be described as below. One version of the prediction model is the ALL model (Al-Qadi Lahouar Leng). With the water component being added to the ALL model, a modified version of the model is named the ACA model (Al-Qadi Cao Abufares). Conveniently, setting the moisture content in the ACA model to zero results in the ALL model. The ALL and ACA models are fundamental models derived based on the dielectric mixing theory. Compared to empirical models, ALL/ACA models can be used for any asphalt mix without the need for calibration-if all the required arguments are known. The present disclosure provides a description of the variables included in both models along with typical values for each variable and the methodology to obtain them.

The ACA model may be expressed as the below equation, and, when MC=0, the ACA model may become ALL model. In some implementations, the moisture content prediction model may be the ACA model, and the asphalt density prediction model may be the ALL model.

$${G}_{mb} = \frac{\frac{{ε}_{AC} - {ε}_{b}}{3 {ε}_{AC} - 2.3 {ε}_{b}} - \frac{1 - {ε}_{b}}{1 - 2.3 {ε}_{b} + 2 {ε}_{AC}}}{\begin{matrix}( 1 + MC ) ( ( \frac{{ε}_{s} - {ε}_{b}}{{ε}_{s} - 2.3 {ε}_{b} + 2 {ε}_{AC}} ) ( \frac{1 - {P}_{b}}{{G}_{se}} ) - ( \frac{1 - {ε}_{b}}{1 - 2.3 {ε}_{b} + 2 {ε}_{AC}} ) \\ ( \frac{1}{{G}_{mm}} ) - MC ( \frac{1 - {ε}_{b}}{1 - 2.3 {ε}_{b} + 2 {ε}_{AC}} - \frac{81 - {ε}_{b}}{81 - 2.3 {ε}_{b} + 2 {ε}_{AC}} ) )\end{matrix}} .$$

G_{mb} is the bulk specific gravity (density) of the asphalt mix. This parameter is usually the prediction target value for newly constructed asphalt pavements. In-situ G_{mb} reflects the quality of pavement construction. G_{mb} is usually divided by the “maximum theoretical specific gravity Gₘₘ,” when only binder and aggregates weight and volume are considered, to represent the percent compaction. To ensure good paving quality and long service life, 93-97% compaction is required. In the target compaction range, G_{mb} typically ranges between 2.200 and 2.600. In the field, this value is measured by extracting cores and testing them in the laboratory (AASHTO T 166) or using nuclear gauges (ASTM D2950/D2950M) at sparse locations. In various embodiments in the present disclosure, this value is predicted “continuously” using the ALL model based on ground penetrating radar (GPR) measurements and asphalt concrete inputs.

ε_{AC} is the dielectric constant of the asphalt concrete. This parameter usually has a value between 4 and 7 depending on the components of the asphalt concrete (e.g., aggregate type, asphalt binder percent, density, and moisture content). This is the main asphalt concrete property calculated utilizing signal processing techniques of collected GPR data. In various embodiments in the present disclosure, GPR signals may be pre-processed and corrected for vibration or antenna height shifting, surface moisture effects, signal instability and thin layer effect (if applicable).

ε_{b} is the dielectric constant of the asphalt binder. There has been extensive research stating that this parameter is usually between 2.6 and 3 for different types of modified and unmodified binders. In various embodiments in the present disclosure, a constant value of 3 based on measurements may be used. It's worth noting that asphalt binder only constitutes around 10% by volume of the asphalt concrete compared to around 85% of aggregates.

εₛ is the dielectric constant of the “stone” or aggregates used in the asphalt concrete. This parameter highly affects the prediction results of the models since aggregates constitute 85% by volume of the asphalt concrete. This value usually ranges between 4 and 8 depending on the minerology and crystal structure of the stones (Quartz vs Limestone). This value usually back-calculated using extracted samples or cores from the field. Recently, some researchers used laboratory prepared samples to calibrate their measurements. Various embodiments in the present disclosure included the establishment of an aggregate dielectric constant database for the state of Illinois, which can be expanded to other states. This may eliminate the need to conduct core calibrations.

MC is the moisture content of the asphalt concrete. This value is zero for newly constructed pavements. However, it is not zero for in-service pavements or cold recycled (CR) pavements. For CR projects, moisture content is an important decision-making parameter. When moisture content drops below a certain threshold (2% in Illinois), the road can be opened for traffic safely without risking premature pavement failure. A typical value for MC ranges between 0-5%. Traditionally, this value is obtained through dry coring or nuclear gauges at sparse locations.

Gₘₘ is the maximum theoretical specific gravity. This value is measured in the laboratory for each asphalt concrete design following AASHTO T 209 and ASTM D 2041 standards. It represents the state of having zero percent air voids in the asphalt mix (only aggregates and asphalt binder). Typical Gₘₘ values range between 2.300 and 2.700. This value is provided by the asphalt plant on the job mix formula sheet.

P_{b} is the percent binder by weight in the asphalt concrete mix (in decimal). This is a mix design parameter provided by the asphalt plant on the job mix formula sheet. This value typically ranges between 4-7%.

Gₛₑ is the effective specific gravity of the aggregates. This is the specific gravity of the aggregates excluding the volume of aggregate surface permeable voids to account for absorbed asphalt by the aggregates. This value ranges between 2.500-2.700 for most coarse aggregate types. It can be calculated based on Gₘₘ and P_{b} based on the following equation (1.02 is the specific gravity of asphalt binder):

$${G}_{se} = \frac{100 - {P}_{b}}{\frac{100}{{G}_{mm}} - \frac{{P}_{b}}{1.02}}$$

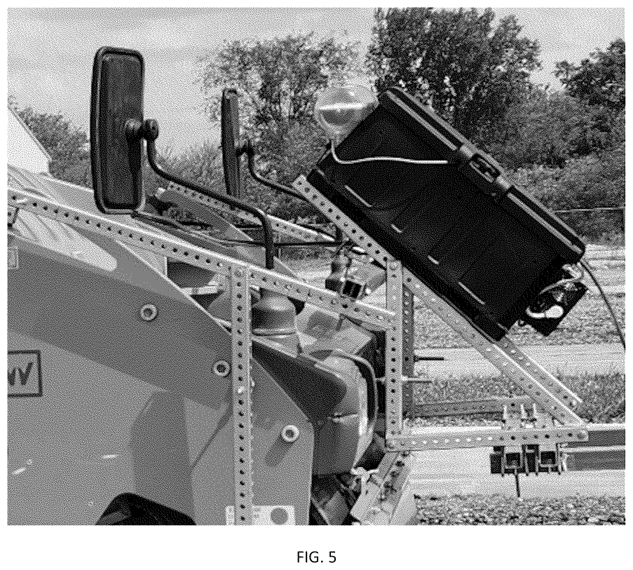

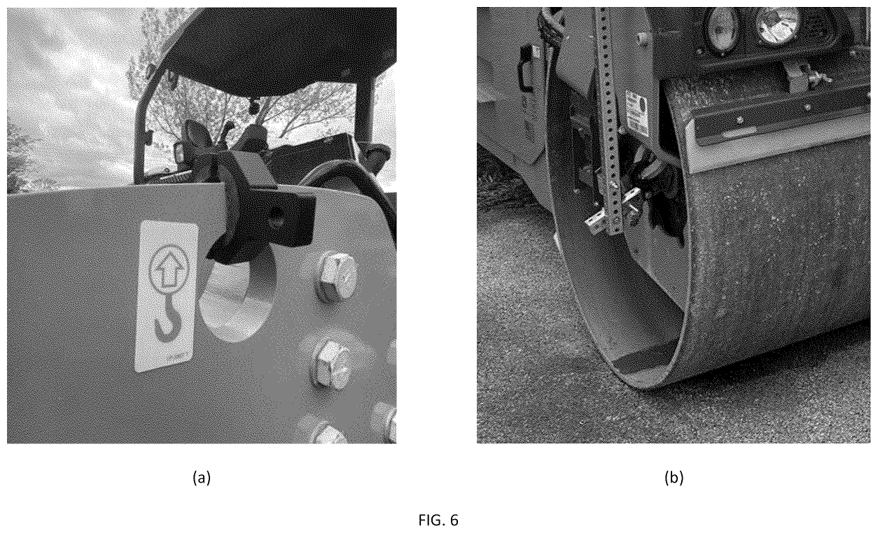

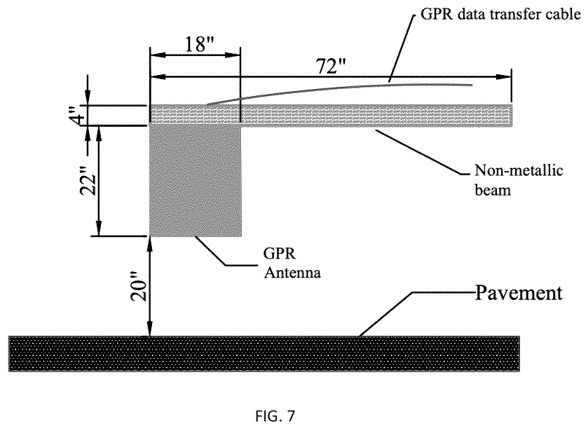

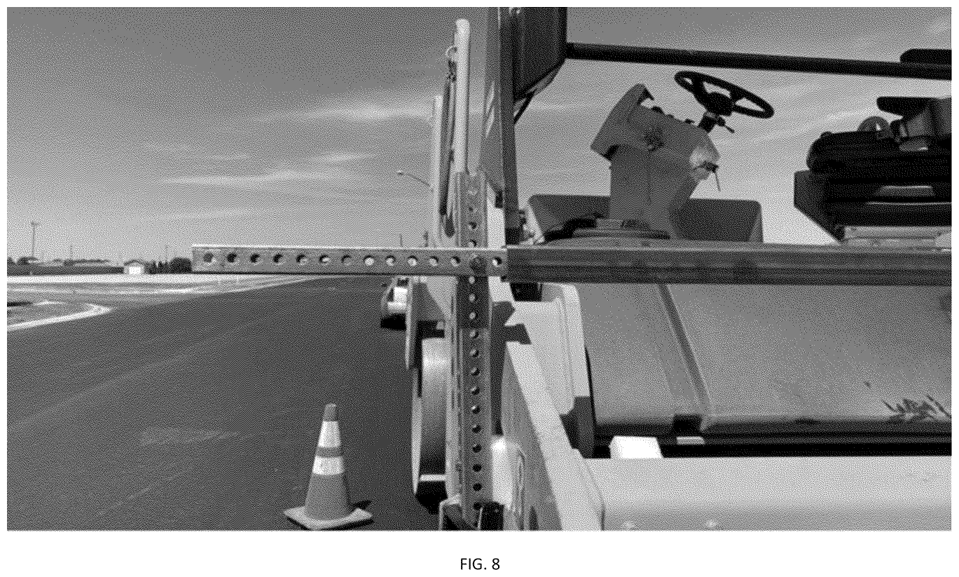

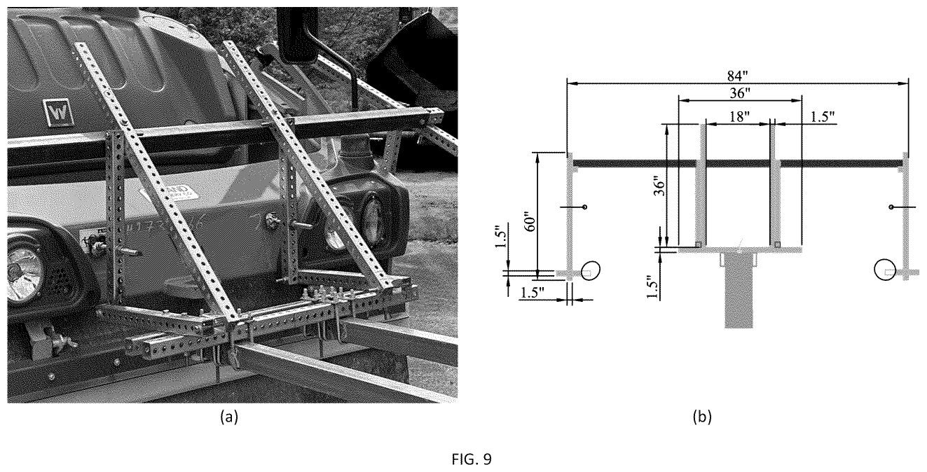

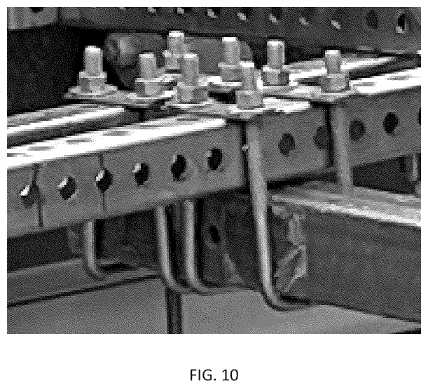

Referring to FIGS. 5-10, the present disclosure provides a detailed description of the GPR-roller compactor mount. FIG. 5 shows a GPS unit attached to the top of the controls box in a thin waterproof container to allow signal transmission. FIG. 6 shows C-clamps connecting the mount to the roller's upper and lower lifting points. FIG. 7 shows an exemplary antenna height and fiberglass beams length. FIG. 8 shows a telescopic steel bar extended to fit another roller type. FIG. 9 shows a truss structure and front view exemplary dimensions in inches. FIG. 10 shows one or more U-clamps connecting the fiberglass beams to the truss structure.

Below, the present disclosure further describes detailed aspects of various embodiments for providing real-time asphalt density and/or moisture content prediction with ground penetrating radar (GPR). The present disclosure describes one or more detailed examples, which does not impose any limitation on the applicability or scope of the present disclosure.

### Signal Stability and the Height-Correction Method for Ground-Penetrating Radar In Situ Asphalt Concrete Density Prediction

Ground-penetrating radar (GPR) has shown great potential for asphalt concrete density prediction used in quality control and quality assurance. One challenge of continuous GPR measurements is that the measured dielectric constant could be affected by signal stability and antenna height. This would jeopardize the accuracy of the asphalt concrete density prediction along the pavement. In this study, signal instability and shifting antenna height during continuous real-time GPR measurements were identified as main sources of error. After using a bandpass filter to preprocess the signal, a least-square adaptive filter, using gradient descent and least mean square methods, was developed to reconstruct the received signal to improve its stability. In addition, simulations were performed to evaluate the impact of geometric spreading caused by shifting antenna height during testing. A height correction was developed using a power model to correct the height-change impact. The proposed filter and height-correction method were assessed using static and dynamic tests. The least-square adaptive filter improved signal stability by 50% and the height-correction method removed the effect of shifting antenna height almost entirely.

Asphalt concrete (AC) pavement density is directly related to pavement structural capacity and service life. It is also an important indicator of AC quality control and quality assurance during AC compaction. An AC density increment of 1% may improve fatigue life and rutting resistance up to 43.8% and 66.3%, respectively, which could lead to a potential 10% service life extension. Given the sensitivity of AC layer density to the compaction effort, it is critical to achieve the tar-get density within a specific window of time.

The traditional way of measuring AC layer density or air voids is to take core samples. This approach is destructive, time-consuming, requires safety measures, and covers only a specific location. Nondestructive methods include using a nonnuclear/nuclear gauge and intelligent compaction (IC). However, nonnuclear/nuclear gauges only estimate in-place density after compaction and cover limited areas. Furthermore, nuclear gauges use radioactive materials, and only licensed operators can transport and operate the device. IC estimates AC pavement density based on AC layer dynamic responses and stiffness. However, application of IC for density measurement does not provide reliable results because AC stiffness changes rapidly with cooling.

Among available nondestructive techniques, ground-penetrating radar (GPR) is deemed to be a cost- and time-effective method that could be used to predict AC density or air voids during the compaction process. Compared with other destructive or nondestructive test methods, GPR has the advantage of potential high-accuracy, large-coverage, and relatively high-speed surveys.

In the AC pavement density measurement, GPR sends electromagnetic (EM) waves into the ground and receives the reflected signals from the interface of two materials with different dielectric properties. The signals can be used to compute the pavement's dielectric properties, which can be then related to AC's densities through theoretical or empirical formulas. Theoretical methods include the Al-Qadi-Lahour-Leng (ALL), Rayleigh, Bottcher, and Lichtenecher-Rother models. Most methods are based on EM mixing theories. Empirical relationships were also built between a material's dielectric constant and density or air voids through regression analyses. The accuracy of measured dielectric constant is essential for evaluating pavement density.

For AC density measurement, the AC pavement material is assumed as dry and lossless. The dielectric constant of a pavement is needed to calculate the pavement's AC density or air voids. In field testing, the reflection amplitude method is widely used because it is easier compared with the common mid-point method and does not require pavement core drilling to obtain the two-way travel time. Thus, it is more widely used in the field. The dielectric constant of an AC pavement layer is decided by the reflected amplitude from the material using the reflection method.

In GPR surveys, the reflected amplitude is affected by transmitted signal instability and antenna height from the pavement surface. For static GPR tests, measured reflected amplitude can be assumed as constant during tests and a mean value can be used to calculate a material's dielectric constant. For continuous tests, however, variation of reflected amplitude is affected by three elements: measured material's heterogeneity, instability of transmitted signal, and shifting antenna height. To predict an AC material's density using the reflection amplitude method, the effects of signal instability and antenna height from pavement surface should be identified. This allows differentiating these effects from the material's heterogeneity impact.

The instability of the transmitted signal may be caused by environmental changes (temperature or humidity variations), improper waveform, internal and external electrical noise, feeding voltage alterations, mechanical vibrations, antenna shielding problems, uncontrolled responses to impedance variations around the antenna, and so forth. The Texas Department of Transportation proposed several methods to ensure short- and long-term signal amplitude: stability, time-interval stability, and signal-to-noise ratio. Based on these methods, Rial et al. proposed a methodology to calibrate GPR devices and to verify proper operation.

Stability analysis is important to understand antenna capabilities and limitations. The American Society for Testing and Materials (ASTM) released standards on GPR application and defined several tests to verify the stability of the GPR signal during the calibration phase and before testing. Benedetto and Tosti later used a reduced Taylor's expansion to assess the performance of the ASTM standard test methods. Among several instability indices, short-term instability has a significant impact on the accuracy of the reflected amplitude measurement. It shows the variation of reflected amplitude from trace to trace.

AASHTO PP98-19 set 0.08 as an acceptable level for dielectric variation in air-coupled dielectric constant measurement. The impact of signal instability on AC density or air void measurement, however, has not been quantified. Research on analyzing the sources of instability or improving signal stability is limited. In this paper, the impact of signal instability on AC density prediction is presented. After using a bandpass filter to preprocess the signal, a least-square adaptive filter was developed to improve signal stability in static and dynamic tests.

During GPR surveys, measurements can be carried out along the road at highway speeds. A previous study shows the measurement error increases as the vehicle speed increases. This is caused by shifting antenna height with respect to the pavement's surface. The antenna height affects the reflection amplitude because of the geometric spreading of transmitted energy, thus affecting measurement accuracy. Several height correction models have been proposed, but the models have not been compatible with each other. Fundamental EM theory must be used to determine the effect(s) of antenna height on GPR measurement to develop a height-correction model. In this paper, a height-correction algorithm was developed to eliminate the measurement error caused by shifting antenna height during GPR continuous dynamic tests. The proposed algorithms for correcting signal instability and shifting antenna height were validated by laboratory tests.

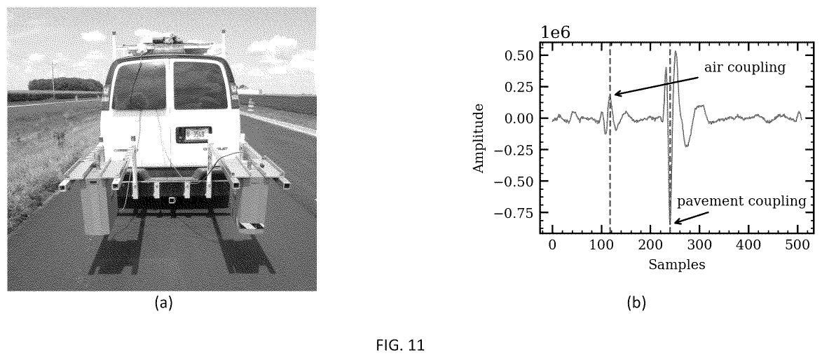

A pavement's dielectric constant can be predicted using the reflection amplitude method (FIG. 11a). Air-coupled antennas are usually set at approximately 0.5 m above the pavement surface. The transmitter antenna sends EM waves into the ground and the receiver antenna receives the reflected waves. From the received signal, at least two pulses can be easily differentiated (FIG. 11b). The first pulse comes from the receiver antenna directly; this pulse is usually called air coupling or direct coupling. The second pulse comes from the reflection waves from the air-pavement interface, which is called pavement coupling. The antenna's height can be calculated using the two-way travel time between direct coupling and pavement coupling. The amplitude of pavement coupling is recorded as Aₚ. A complete perfect electricity conductor, usually a 4*4 ft² copper plate to cover the antenna's footprint, is placed beneath the antenna on the pavement's surface. The amplitude of the reflector coupling (copper plate) is recorded as A_{c}. The dielectric constant ε of the pavement surface is calculated using the following equation.

$$ε = {( \frac{1 + \frac{{A}_{p}}{{A}_{c}}}{1 - \frac{{A}_{p}}{{A}_{c}}} )}^{2}$$

To measure pavement density or air voids using the reflected amplitude method, reflection amplitudes on AC pavement and the copper plate must be measured at the same height. For the same material under measurement, the higher the antenna to the tested material, the more energy dissipates in the air and the lower the reflection amplitude.

After the pavement surface dielectric constant is deter-mined, the AC pavement density can be calculated using the ALL model, shown in the equation below. The model builds a relationship between the AC mixture dielectric constant (ε_{AC}) and bulk specific density (G_{mb}):

$${G}_{mb} = \frac{\frac{{ε}_{AC} - {ε}_{b}}{3 {ε}_{AC} - 2.3 {ε}_{b}} - \frac{1 - {ε}_{b}}{1 - 2.3 {ε}_{b} + 2 {ε}_{AC}}}{\frac{{ε}_{s} - {ε}_{b}}{{ε}_{s} - 2.3 {ε}_{b} + 2 {ε}_{AC}} ( \frac{1 - {P}_{b}}{{G}_{se}} ) - \frac{1 - {ε}_{b}}{1 - 2.3 {ε}_{b} + 2 {ε}_{AC}} ( \frac{1}{{G}_{mm}} )}$$

Where Gmm is the maximum specific gravity of AC mixture,
Gse is the effective specific gravity of aggregates,
Pb is the binder content of AC mixture,
εb is the dielectric constant of asphalt binder, and
εs is the aggregate dielectric constant.

These AC volumetric values can be obtained from the plant before pavement compaction or from a database in the case of the aggregate. Air voids can be derived from Gₘₘ and G_{mb} using the following equation:

$$Air void \% = \frac{{G}_{mm} - {G}_{mb}}{{G}_{mm}} * 100 \%$$

During GPR measurements, the reflection amplitude is not a constant value. During GPR measurements, the reflection amplitude is not a constant value. As described earlier, the mean amplitude is a function of the AC mixture's volumetric properties. In addition, there is a random variation caused by instability. An instability index J is defined to evaluate the short-term amplitude variation of the reflected signal. According to ASTM standards, the antenna is positioned at its far-field distance from a reflector plate. The GPR unit is turned on and allowed to operate for a 20-min warm-up period. After warming up, 100 traces are recorded at the maxi-mum data acquisition rate. The instability of the signal is evaluated as:

$$J = \frac{( {A}_{max} - {A}_{min} )}{{A}_{avg}} < 0.01 ( 1 \% )$$

Where
Aₘₐₓ is the maximum plate reflection amplitude for all 100 traces,
Aₘᵢₙ is the minimum metal plate reflection amplitude for all 100 traces, and
A_{avg} is the average trace amplitude of all 100 traces.

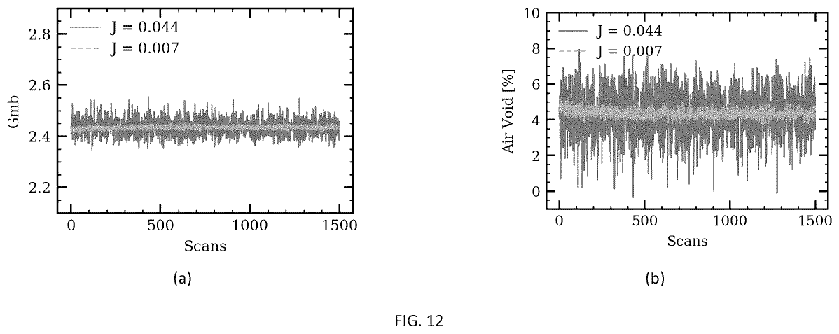

The signal stability test results for the GPR system should be less than or equal to 1%. To illustrate the impact of signal instability on the predicted AC density and air voids, a comparison was made between the signal with the instability index of 4.4% and with the instability index of 0.7% in FIG. 12. The tests were both static, and the instability indices were observed from two different 2 GHz antennas currently used in the market. The dielectric constant was measured by reflection amplitude method, and the density and air voids were calculated using the previous equations.

FIG. 12 shows that a higher instability index indicates a higher signal amplitude variation and air-void variation. For J=4:4%, air voids change from 0% to 8%, while for J=0:7%, air voids only vary between 4% and 5%. Both tests were taken under the static condition, meaning the same pavement area is measured. The AC density or air voids measured change largely from scan to scan when the instability index is high. This is a problem in continuous GPR tests that has been overlooked. Pavement density may not be accurately predicted because of signal instability and antenna height shifting that affect the reflected amplitudes. Although traditional methods, such as moving average and scan stacking, filter high frequency information, they do not improve the accuracy of pavement density prediction. In this study, the effects of signal instability and antenna height from pavement surface are determined and eliminated. This allows the effect of AC material's heterogeneity impact on predicted dielectric constant be pronounced. In this study, the effects of signal instability and antenna height are determined and eliminated to focus on the effect of the material's heterogeneity that affect the AC dielectric constant.

Signal instability shows as variation of the reflection (pavement-coupling) amplitude from the pavement surface. A bandpass filter was used to preprocess the received signal from the pavement surface. A least-square adaptive filter was developed to reconstruct the received signal and improve signal stability. A 2-GHz antenna from Geophysical Survey Systems, Inc. (GSSI) was used in this study.

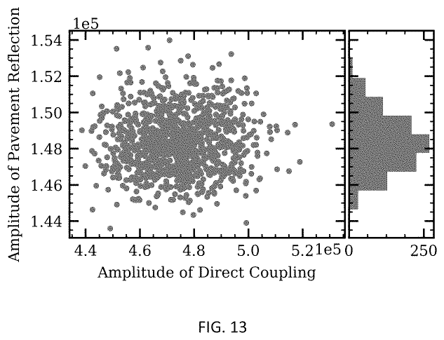

Characteristics of Signal Instability: The relationship between air- and pavement-coupling amplitude and the distribution of pavement-coupling amplitude are shown in FIG. 13. The pavement-coupling amplitude is normally distributed. The correlation coefficient between air- and pavement-coupling amplitudes is close to 0, showing there is no correlation between the two amplitudes. This suggests that signal instability is not caused by unstable voltage input. If signal instability is caused by unstable voltage input, then the amplitudes of air and pavement couplings will be either high or low simultaneously, as the effect will be on both.

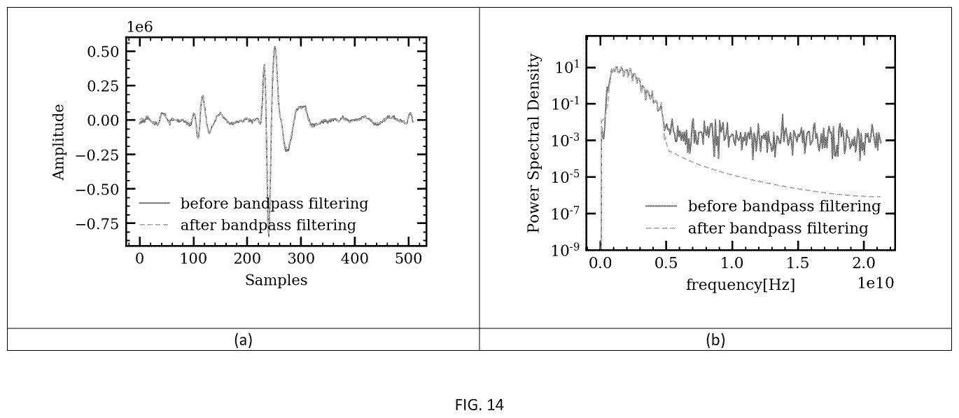

Bandpass Filter Application: Environment noises and internal problems with the electric device can cause signal instability. Internal problems with the electric device include instability in the reconstruction of the signal from the recorded samples, such as analog to digital quantization error and fair sampling clock performance. A bandpass filter was used to preprocess the received signal. For a 2-GHz antenna, the high-pass cutoff frequency was set at 0.45 GHz and the low-pass cutoff frequency was set at 5 GHz. This would eliminate most environment noise without losing signal information. The received signal and its power spectrum density (PSD) are shown in FIG. 14. The instability index of the signal before using bandpass filtering is 5.4%, and the instability index of the signal after using bandpass filtering is 3.8%. Bandpass filtering can decrease the signal's instability index to some extent. However, to further improve signal stability, an advanced signal-processing technique needs to be applied on the received signal.

The shape of the time-domain signal is important for GPR. The waveform of the transmitted signal can affect the resistance of the signal to surrounding or internal noises. Signals with zero phase in frequency domain are more resistant to phase distortion than non-zero-phase signals. A Ricker wavelet has a higher signal-to-noise ratio and spatial resolution as well as a lower power loss than other wavelet shapes, such as Gaussian wavelet, Sine wavelet, and Raised cosine.

Pulse Shaping Using Adaptive Filter. Pulse shaping is a signal-processing technique of changing the waveform of trans-mitted pulses to an ideal waveform. It is usually used for the optimality of radar system performance. Least-square adaptive filters, using gradient descent and least mean square error methods, respectively, were developed to reshape the original pulse wave-form and to improve signal stability.

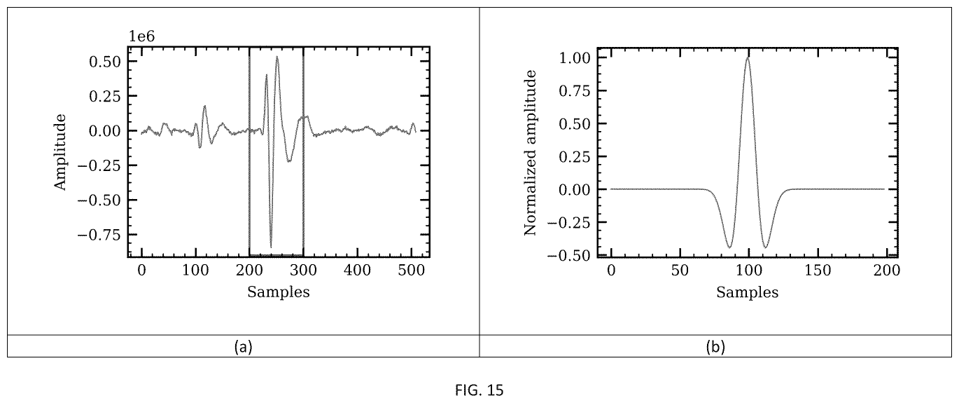

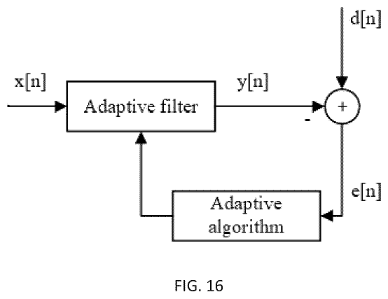

The original transmitted pulse x[n] can be achieved by the reflected signal on a copper plate (FIG. 15a). The ideal waveform, sometimes called reference waveform, d[n] is chosen as a Ricker wave in this study (FIG. 15b). The idea is to pass the input signal x[n] through a filter to formulate an output signal y[n] that is close to the reference signal d[n]. This process can be done iteratively using an adaptive filter, which is illustrated in FIG. 16. The difference between the reference/ideal signal and output signal is error e[n]. The adaptive filter changes its filter coefficients w[n] by an adaptive algorithm to minimize errors. The filtering process can be represented as a convolution process, where * means convolution:

$$y [ n ] = x [ n ] * w [ n ]$$

$${\sum }_{k = 0}^{N - 1} w [ k ] x [ n - k ] = {w}^{T} {x}_{n} , n = 0 , 1 , 2 , …$$

xₙ=(xₙ, . . . , x_{n-N+1})^{T} and w=(w₀, . . . , w_{N-1}) are vectors of the transmitted signal and filter coefficients. N is the length of the filter. The optimum w* is achieved when the mean square error is minimum:

$${w}^{*} = arg min {\sum }_{t = 1}^{T} {( {d}_{t} - {w}^{T} {x}_{t} )}^{2}$$

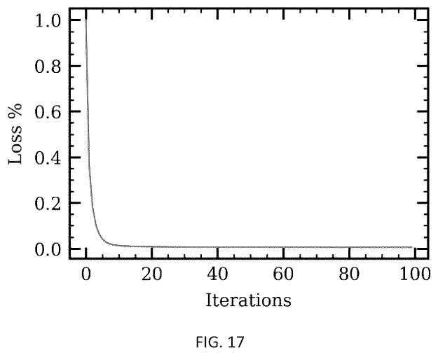

Gradient descent is used to solve this optimization problem. The filter parameters of the next step are a function of previous filter parameters and the derivatives of mean square error between the output and desired signals as follows:

$${w}_{t} = {w}_{t - 1} - \frac{1}{2} μ \frac{\partial {‖ d - Xw ‖}_{2}^{2}}{\partial w} = {w}_{t - 1} - μ {X}^{T} ( d - X {w}_{t - 1} )$$

where μ is step size. The iteration ends when the error converges (FIG. 17). The filter coefficients converge rapidly after the first 100 iterations.

Another approach to optimize the filter coefficient is to calculate the analytical solution of w*, which is called the least mean square method. The derivative of the mean square error with respect to the filter coefficient is set to 0 (in the following equation). M is the length of the transmitted signal.

$$\frac{\partial {‖ e ‖}^{2}}{\partial w} = \frac{\partial {\sum }_{n = 0}^{M - 1} {e [ n ]}^{2}}{\partial w} = 0$$

For each component of the gradient vector,

$$\frac{\partial {\sum }_{n = 0}^{M - 1} {e [ n ]}^{2}}{\partial {w}_{k}} = 2 {\sum }_{n = 0}^{M - 1} e [ n ] \frac{\partial }{\partial {w}_{k}} [ d [ n ] - {\sum }_{l = 0}^{N - 1} w [ l ] x [ n - l ] ] = 2 {\sum }_{n = 0}^{M - 1} e [ n ] x [ n - k ]$$

the righthand of the equation can be further expand as follows:

$${\sum }_{n = 0}^{M - 1} e [ n ] x [ n - k ] = {\sum }_{n = 0}^{M - 1} [ d [ n ] - {\sum }_{l = 0}^{N - 1} w [ l ] x [ n - l ] ] x [ n - k ] = 0$$

$${\sum }_{n = 0}^{M - 1} d [ n ] x [ n - k ] = {\sum }_{l = 0}^{N - 1} w [ l ] {\sum }_{n = 0}^{M - 1} x [ n - l ] x [ n - k ]$$

This can be written in a matrix form:

$${A}^{T} d = ( {A}^{T} A ) w$$

where A is the Toeplitz matrix, and d and w are the vectors of reference pulse and filter coefficients, respectively.

$$A = ( \begin{matrix}\begin{matrix}x [ 0 ] & 0 \\ x [ 1 ] & x [ 0 ]\end{matrix} & … & \begin{matrix}0 \\ 0\end{matrix} \\ \begin{matrix}⋮ & ⋮\end{matrix} & ⋱ & ⋮ \\ \begin{matrix}x [ M - 2 ] & x [ M - 3 ] \\ x [ M - 1 ] & x [ M - 2 ]\end{matrix} & … & x [ M - N + 1 ]\end{matrix} )$$

Thus, the optimum filter is as follows:

$${w}^{*} = {( {A}^{T} A )}^{- 1} {A}^{T} d$$

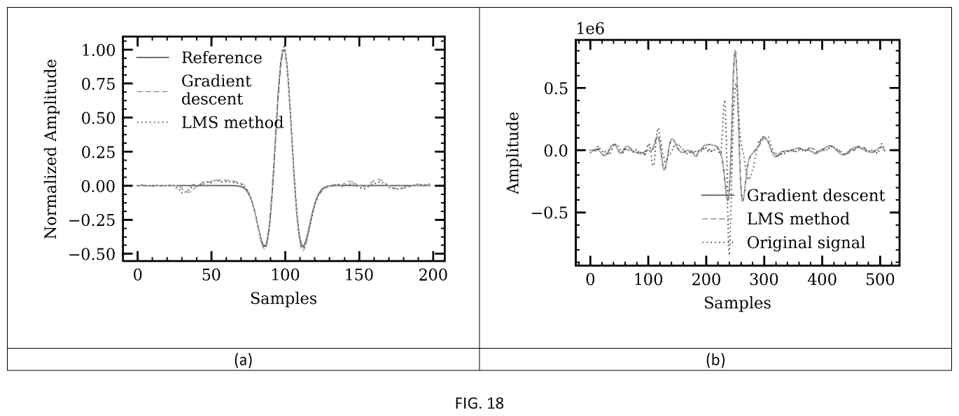

The output signals using gradient descent and least mean square methods are shown in FIG. 18a. The results from the two methods overlap and are close to the reference waveform, except minor differences before and after the pulse. The minor differences do not affect the result, because only the peak value of the pulse is used in the reflection amplitude method. The convolution of the developed filter with the entire scan on the copper plate is shown in FIG. 18b. The instability index of the scan decreased from 5.4% to 2.7% after filtration. The performance of the adaptive filters is similar when using the two methods. However, the gradient descent method is fivefold more time efficient the least square method; hence the former is preferable in practice. No loss was found in two-way travel time or relative amplitude after filtration.

A received GPR signal may be interpreted as a convolution of a reflectivity series r[n] and a transmitted signal x[n] plus some superposed noise N[n]. After the convolution with the designed filter, the resulted signal is w[n]*x[n]*r[n]+N′[n]. Because the convolution operation is associative, the signal is equivalent as d[n]*r[n]+N′[n]. It is the same as the convolution of the ideal transmitted signal and the reflectivity series. The filter can be further used to improve signal stability on other tests using the same antenna.

For continuous GPR tests, another source of inaccurate results is the shifting height of the antenna mounted on a vehicle. Antenna height variation impacts the received signal energy intensity and, thus, the amplitude. Because air is considered a nonabsorbent media in most GPR studies, the cause of attenuation during the propagation of energy is mainly due to geometric spreading. The need for a height-correction is well known in the industry. Although height correction on GPR equipment has been applied by some vendors, such corrections have not been published. Several height correction models from literature were discussed. A power fit model is proposed herein based on simulation results.

Simulation: According to the EM theory, for a single electric dipole, the electric field intensity is a function of the distance to the dipole. The electrical field is divided into the near-field and far-field regions as well as transition zone in terms of distance from the center of radiation. Distance from two wavelengths to infinity is regarded as far field. The electric field intensity is proportional to the inverse of distance from the radiation center. This relationship is for a single dipole and neglects effects of the waveguide shape and antenna gain in specific directions.

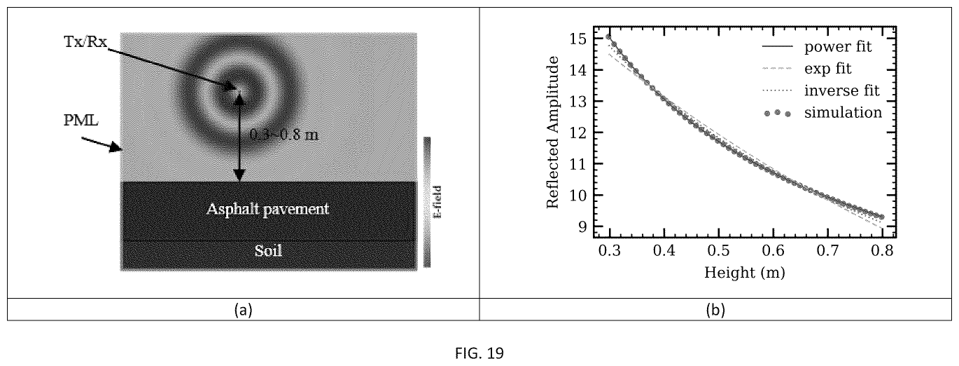

A simulation was developed in gprMax2D to illustrate the effect of height shift to the dielectric constant measurement using the finite-difference time-domain method. The model configuration is shown in FIG. 19a. Tₓ/Rₓ were the dipole antenna's transmitter and receiver. A perfectly matched layer (PML) was used to absorb the EM waves to simulate open boundary problems. A Ricker wave was used as the source wave in the simulation. The height of the antenna varied from 0.3 m to 0.8 m above the pavement surface. The relationship between the reflected peak amplitude and antenna's height is shown in FIG. 19b. The height of the antenna was calculated using the two-way travel time between direct coupling and pavement coupling.

Several height-correction models were presented in the literature to adjust the geometric spreading loss. The first is the inverse model: A=a/(a+Dₜ) for 1 GHz antenna, where a=1.4678, b=43.832, A is the corrected amplitude, and Dₜ is the travel time between the direct-coupling and pavement-surface-coupling peaks. This model has caused essential incorrectness in both 1 GHz and 2 GHz antennae. In addition, parameters a and b needed to be found from regression curves. The second is the power model: A=ah^{b}, where h is the height of the antenna and a and b can be derived from regression. The third is exponential model: A=ae^{bh}, where h is the height of the antenna and a and b can be derived from regression curves. The fitted curve using the three simulation methods are shown in FIG. 19b. The regression parameters are shown in the following table:

| | Model Types | a | b | R² |
|---|---|---|---|---|
| 2D | Inverse model | 0.0840 | 0.0426 | 0.9960 |
| | Power model | 8.3331 | −0.4890 | 1.0 |
| | Exponential | 19.3438 | −0.9663 | 0.9842 |
| | model | | | |

The inverse and power models have a higher R² value than the exponential model. For the inverse model, however, the parameters a and b change when the signal excitation changes. This may cause problems in the field when different antennas are used. If the theoretical power parameter b=−0.5 is used in a 2D simulation, then the R² value is equal to 0.9995, which also fits the data well.

Once the amplitude is corrected, the dielectric constant calculated from the reflection amplitude method can also be corrected as:

$${ε}^{′} = {( \frac{1 + \frac{{A}_{p}^{′}}{{A}_{c}}}{1 - \frac{{A}_{p}^{′}}{{A}_{c}}} )}^{2} = {( \frac{1 + \frac{{A}_{p}}{{A}_{c}} {( \frac{{h}_{c}}{{h}_{p}} )}^{b}}{1 - \frac{{A}_{p}}{{A}_{c}} {( \frac{{h}_{c}}{{h}_{p}} )}^{b}} )}^{2}$$

where A′ₚ is the corrected amplitude. In field tests, A_{c} is measured statically before the continuous test, so it can be assumed constant during the tests. h_{c} is the reference height, which is the height of the antenna when A_{c} is measured. The reflected amplitude Aₚ from pavement surface is affected by antenna height hₚ in each scan.

Compared with the original dielectric constant equation, the relationship between the corrected dielectric constant and original dielectric constant is:

$${ϵ}^{′} = {( \frac{1 + \sqrt{ϵ} + ( \sqrt{ϵ} - 1 ) c o r r}{1 + \sqrt{ϵ} - ( \sqrt{ϵ} - 1 ) c o r r} )}^{2} , where corr = {( \frac{{h}_{c}}{{h}_{p}} )}^{b}$$

The error between the corrected and uncorrected dielectric constant is calculated as DC error=ε′−ε.

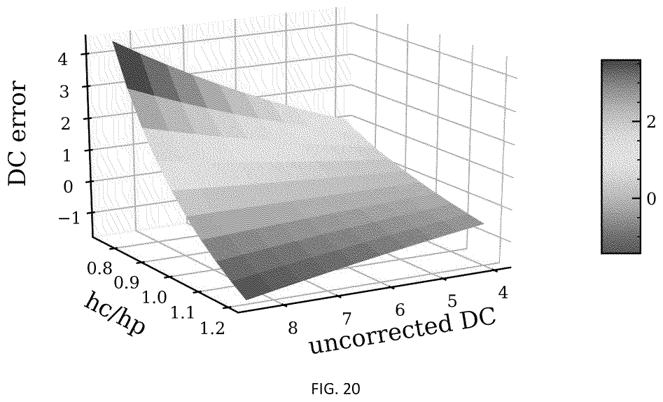

The relationship among the dielectric constant error, h_{c} to hₚ ratio, and uncorrected dielectric constant is shown in FIG. 20. The higher the uncorrected dielectric constant, the higher the dielectric constant error. The higher the dielectric constant, the more vulnerable it is to shifting antenna height.

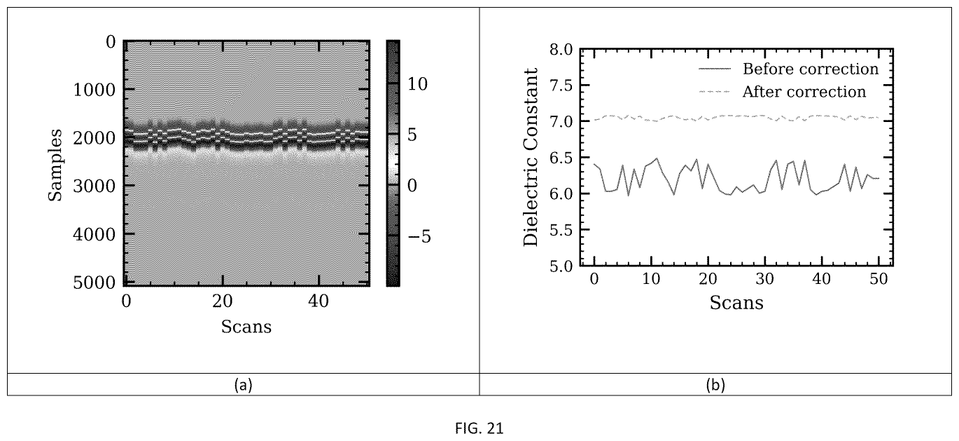

To demonstrate the effect of shifting antenna height on the dielectric constant calculations, a simulation of a vibrating antenna was performed in gprMax2D. The antenna's height changed within 0.5±0.02 m randomly from the pavement. The dielectric constant of the pavement was set to 7.0. The simulation result is shown in FIG. 21. The reference height h_{c} was chosen as 0.45 m. The dielectric constant of the pavement was back-calculated using the original equation before the correction. The uncorrelated dielectric constant has a relatively high variation and deviates from the input dielectric constant. The corrected dielectric constant is very close to the input value 7.0.

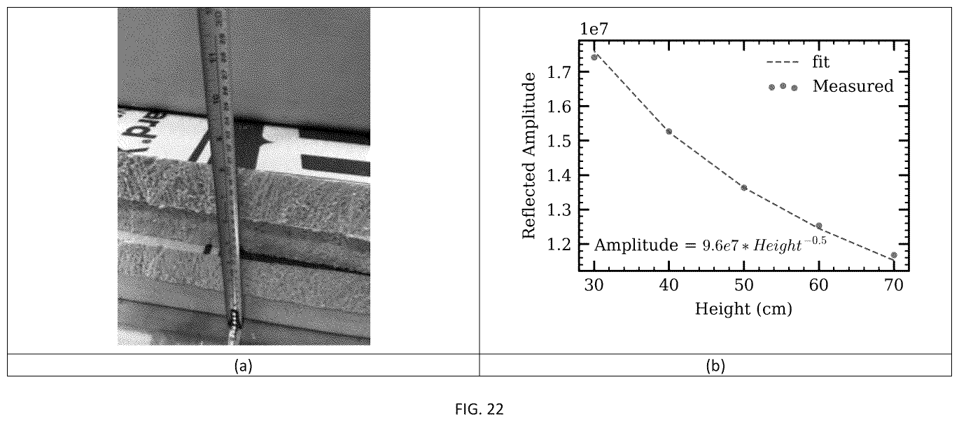

Laboratory Tests: Laboratory tests were performed to validate the power parameter b. Styrofoam with a known dielectric constant (equal to 1) was inserted between the antenna and copper plate (FIG. 22a) to adjust the height of the antenna. The relationship between reflected amplitude and height is fitted using power parameter b=−0.5 (FIG. 12b). The R² value for the relationship between reflection amplitude and height is 0.997.

The accuracy of the proposed height correction algorithm relies on the determination of the positions of air coupling and pavement coupling. Possible error may come from 1) noise in the air coupling; and 2) pavement coupling overlapping with reflection from the next layer interface. For both cases, the noise or signal overlapping has less effect on the position of target signal than on the amplitude value. From the observation in lab and field tests, the offsets of air/pavement coupling signal are less than five samples. Considering the common sampling rate of 512 (sample per scan) in the field, the error of antenna height measurement caused by signal position changing is within 2%. Taking the height error of ±2% and using it in the correction equation, the error of corrected dielectric constant is within ±1%, which is acceptable.

Test validation and discussion: Two sets of tests were performed to validate the proposed algorithms for signal stability improvement and height correction. For the static test, the proposed least-square adaptive filter was used, and signal stability was compared before and after filtration. For the dynamic test, a bumping test was performed to induce height shifting of antennas manually. Both the least-square adaptive filter and height-correction method were used. The calculated air voids were compared before and after applying the algorithms.

Stability Improvement in Static Tests: Static tests were conducted to evaluate the performance of the least-square adaptive filter on signal stability. A 2G commercial horn antenna and control unit were used in the study. In the test setting, scan rate indicates the number of scans the antenna collected per second. When the scan rate per second is low, the control unit automatically performed sample stacking, which means the antenna can collect multiple samples along the same scan simultaneously. This improves the signal-to-noise ratio and partially improves signal stability. This is usually not recommended in field tests, because fewer scans are collected along the pavement in the low scan rate. Thus, less information about the pavement is obtained. This technique, however, was used in the static test to evaluate the performance of the proposed filter with different original signal stabilities. The following table shows the instability indices of the signals for various sampling and scan rates. Sampling rate means the number of collected samples along each scan.

| | | Scan rate (Hz) | | | | | | |
|---|---|---|---|---|---|---|---|---|
| Sampling rate | | 20 | 40 | 60 | 80 | 100 | 120 | 140 |
| 512 | Before | 1.597% | 2.469% | 3.305% | 3.384% | 4.283% | 5.375% | 5.861% |
| | correction | | | | | | | |
| | After | 0.889% | 1.090% | 1.499% | 1.617% | 2.353% | 2.530% | 2.539% |
| | correction | | | | | | | |
| 1024 | Before | 1.791% | 2.617% | 3.611% | 3.778% | 3.671% | | |
| | correction | | | | | | | |
| | After | 0.993% | 1.640% | 2.481% | 2.373% | 1.928% | | |
| | correction | | | | | | | |

Signal stability improves with decreased scan rate. For various original signal instability indices, the least-square adaptive filter reduced the instability index to almost half of the original index; the signal stability is improved by 50%. This decreases the variation of the measured dielectric constant and further improves the accuracy of the calculated pavement density and air voids.

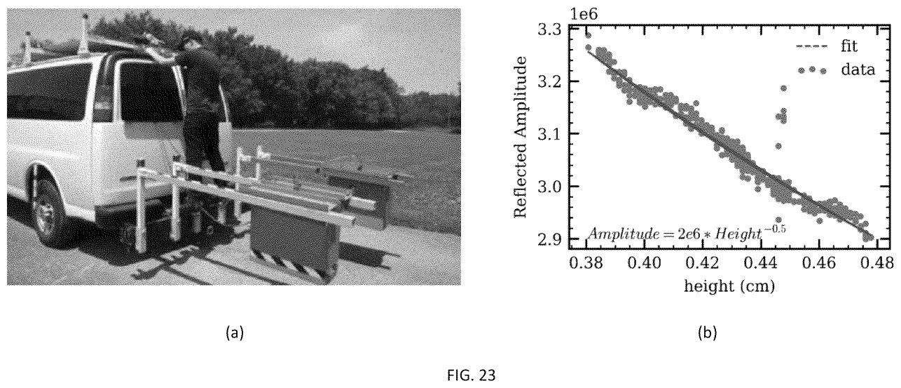

Stability Improvement and Height Correction in Dynamic Tests: A dynamic test was designed to evaluate the performance of the height-correction algorithm FIG. 23a shows the test. Antennas were mounted at the back of a van. The van was static, and a person jumped on the bumper of the van to induce height variation of the antennas periodically, simulating the impact of a vehicle bumping at high-speed GPR surveys. Because the van did not move, the antenna always measured the same pavement surface area. This excludes the effect of material heterogeneity on the test results. Only signal instability and shifting antenna height cause the variation of predicted air voids.

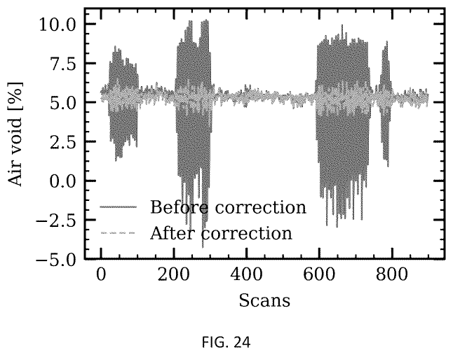

After filtration using the least-square adaptive filter, the relationship between reflected amplitude and height was obtained from the dynamic test (FIG. 23b). Height was calculated from the two-way travel time between direct coupling and pavement coupling. Except some outliers, the relationship can be fitted well by the power model. Assuming Gₘₘ=2.545, Gₛₑ=2.741, P_{b}=0.051, e_{b}=3, eₛ=7, air voids were calculated using the ALL model and air void equations. The calculated air void before and after the height-correction method are shown in FIG. 24.

Because the antenna measures the same location, the variation of measured air voids comes from signal instability and shifting antenna height. Air-void variation before the correction is caused by the person jumping on the bumper. Before the height correction, the calculated air voids vary from 4% to 10%. The negative air voids come from the out-of-range dielectric constant. After the correction, the air voids fluctuate around 5.5%, and the air-void variation caused by jumping is at a similar level to the air voids measured statically. The proposed algorithm was shown to remove the effect of shifting antenna height almost entirely.

Conclusions: A continuous GPR survey has been used for in situ AC pavement density measurements for quality control and quality assurance. However, the calculated dielectric constant is jeopardized by signal instability and shifting antenna height, resulting in AC density error prediction. A least-square adaptive filter is proposed that improves GPR signal stability and a height-correction method is introduced to remove the effect of antenna shifting during dynamic tests. The proposed filter and height correction were assessed by static and dynamic tests. The developed adaptive filter improves signal stability by approximately 50%. The introduced algorithm for controlling the impact of height with respect to pavement surface removes the effect of shifting antenna height. The proposed algorithms are expected to be applied to field data for near real-time dielectric constant prediction. The application of the algorithms is currently being validated.

### Model Development for Moisture Content and Density Prediction for Non-Dry Asphalt Concrete Using GPR Data

Ground-penetrating radar (GPR) is a non-destructive testing technique used to assess various civil structures, including pavements. It may be applied to predict asphalt concrete (AC) layer thicknesses and dry densities. Because moisture may exist in in-service AC pavement layers and hinder the density prediction, quantifying moisture content in AC would improve its layer-density prediction. In addition, quantifying moisture content of cold recycling would allow monitoring of the curing process of the treatment. Hence, the proper time for opening roads to traffic and/or placing an overlay could be identified. In this study, data were collected from both field cold-recycling projects and laboratory test slabs. The combined dataset was used to correlate measured moisture content to the dielectric constant of AC mixes. The Al-Qadi-Cao-Abufares (ACA) model was derived based on the electromagnetic mixing theory. This model is a modification to the Al-Qadi-Lahouar-Leng (ALL) model; it incorporates moisture's effect on the bulk dielectric constant to predict non-dry AC density. The model predicts non-dry AC density with an average error of 2% and also predicts moisture content with a root mean square error of 0.5%.

The material of interest in this study is asphalt concrete (AC). To minimize the energy consumption and environmental impact of AC, the following may be considered: (1) introduce new sustainable materials, (2) improve production and construction techniques, (3) recycle AC pavements, and (4) improve periodic assessment and evaluation of AC roads. Cold recycling (CR) is one rehabilitation technique that has been gaining popularity due to its environmental and economic benefits, including cold in-place (CIR) and cold central-plant recycling (CCPR) practices. A well-compacted CR mixture typically has 8% to 16% voids. Hence, an overlay is usually required to protect the mixture from moisture intrusion. However, for pavements with low-volume traffic, a surface treatment (e.g., chip seals, slurry surfacing, or micro-surfacing) has been used.

To facilitate mixing and placement of aged, reclaimed asphalt pavement (RAP) in CIR/CCPR, a recycling additive; emulsions, or foamed asphalt is usually added. Asphalt emulsion is a stable dispersion of asphalt-cement droplets in water; it contains asphalt binder, water, and emulsifying agents. Once an asphalt emulsion is mixed with aggregates, the water in the emulsion begins to separate from the asphalt binder and behaves as free water. The emulsion acts as an asphalt binder after curing; the process of evaporation of free water. Hence, curing is a critical step in CR treatment to achieve adequate structural capacity before opening the road to traffic or placing an overlay. Emulsion curing rate depends on multiple factors, such as the nature of the stabilization (particularly if asphalt emulsion is used), ambient temperature, humidity, and moisture content of the mixture. In addition, compaction level and drainage characteristics affect curing rate.

Ground-penetrating radar (GPR) is a non-destructive testing technique that has been used for pavement evaluation for the past four decades. It is a system that emits electromagnetic (EM) waves and receives the waves' reflections. For pavements, when the emitted EM waves encounter a discontinuity—such as a different material type or a flaw—portions of the waves are reflected. The reflected signals may be analysed using signal-processing techniques to extract meaningful information about the assessed system. A material difference or a discontinuity is indicated by the change in dielectric properties. GPR has been successfully used on AC pavements for a multitude of applications, including the estimation of AC-layer thicknesses, predicting AC-layer density, detecting moisture in AC, and assessing the effectiveness of drainage systems. Real-time density monitoring during compaction is another promising application of GPR. Advanced signal-processing techniques have been developed for such applications.

When moisture is free (not bound to the emulsion), it masks the reflected signals, making them appear higher in amplitude, thus resulting in overestimated density. This masking is due to the fact that the dielectric constant of water is 78 at an ambient temperature of 73° F. (23° C.; this dielectric constant changes slightly with temperature and is usually considered to be 81). Clearly, the water's dielectric constant is significantly different from the typical dielectric constant of AC, which ranges from 4 to 10, depending on the aggregate type used. The contrast in the dielectric properties of the components was previously used between water and soil materials to determine partial water saturation (up to 60%) using the square root formula for soils.

Moisture estimation, using GPR data, is dependent on the percent of air voids and their connectivity. If the pavement structure is highly porous, water drains and does not affect GPR measurements. In contrast, if the AC mixture has a few connected voids, moisture is trapped on the surface and highly affects the GPR surface-reflection amplitude. A significant challenge for applying GPR to CR mixes is the presence of bound water within the emulsion before it breaks (cures). Presence of bound water makes moisture detection difficult because the dielectric constant of free water is an order of magnitude higher than that of bound water.

This study introduces a new model to predict non-dry AC-layer dry density—the Al-Qadi-Cao-Abufares (ACA) model, which may also be used to predict moisture content in AC layers. This model will help pavement engineers monitor AC density and/or moisture content as part of the important quality-control/quality-assurance practices. Two methodologies were used in this study: field and laboratory tests. Field tests were performed on CR projects, while laboratory tests were performed on slabs in a laboratory-controlled environment.

Field Tests: In total, six field projects were visited, all were conducted on minor arterial roads in Illinois: five CIR projects and one CCPR project. Four of the CIR construction sites and the CCPR site were visited and tested between May 2020 and September 2020; the same tests were repeated on one more CIR project in July 2021. The CIR projects were performed on IL-91, IL-116, IL-100, and IL-61 in Peoria and on IL-64 in Ogle; all used engineered emulsions as the recycling agent. The CCPR project was performed on RT-509 in Indianola. All CIR-treatment depths were 4 in. (10.2 cm), and CCPR treatment depth was 3 in (7.6 cm). Field data were collected during construction, including GPR scans, sand-cone measurements, and loose samples after final roller compaction of test locations. Because weather affects CR curing, weather information was documented hourly. The following table shows an example of the testing schedule for the IL-61 project.

| | Location # | | | | | | |
|---|---|---|---|---|---|---|---|
| | 1 | 2 | 3 | 4 | 5 | 6 | 7 |
| | Station | | | | | | |
| | 848 + 00 | 849 + 50 | 851 + 00 | 852 + 50 | 1034 + 00 | 1032 + 50 | 1031 + 00 |
| | SB | SB | SB | SB | NB | NB | NB |
| | Transverse | | | | | | |
| | LWP | RWP | LWP | RWP | LWP | CL | RWP |
| Aug. 12, 2020 | 0 h | 0 h | 0 h | 0 | N/A | N/A | N/A |
| Aug. 13, 2020 | 1 D | 1 D | 1 D | 1 D | 0 h | 0 h | 0 h |
| Aug. 14, 2020 | N/A | N/A | N/A | N/A | N/A | N/A | N/A |
| Aug. 15, 2020 | N/A | N/A | N/A | N/A | N/A | N/A | N/A |
| Aug. 16, 2020 | N/A | N/A | N/A | N/A | N/A | N/A | N/A |
| Aug. 17, 2020 | 5 D | 5 D | 5 D | 5 D | 4 D | 4 | 4 D |
| Aug. 18, 2020 | N/A | N/A | N/A | N/A | N/A | N/A | N/A |
| Aug. 19, 2020 | 7 D | 7 D | 7 D | 7 D | 6 D | 6 D | 6 D |

*SB stands for southbound, and NB stands for northbound.
**LWP stands for left wheel path, RWD stands for right wheel path, and CL stands for centerline.

The GPR static tests were performed, as shown in FIG. 1, using two air-coupled horn antennas (Geophysical Survey Systems, Inc.) at a central frequency of 2.0 GHz. Prior to the GPR survey, the GPR system was turned on and left to warm up for 20-30 min. A distance-measuring instrument (DMI) and a global position system (GPS) were connected and calibrated. Time mode was selected for the GPR survey. The duration of each scan was selected to be about one minute.

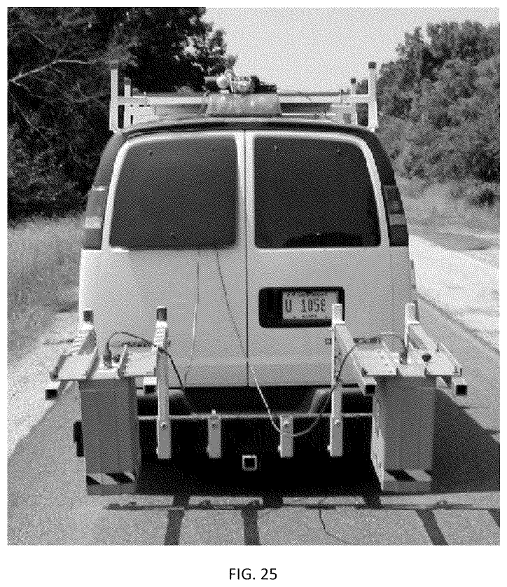

GPR static-test spots were marked using orange spray paint (FIG. 25) to allow repetitive monitoring of the same locations. Then, sand-cone holes were made close to the GPR test spots; and samples were collected within proximity but far enough away so as not to impact the later GPR measurements. The collected samples penetrated through the entire CR layer thickness.

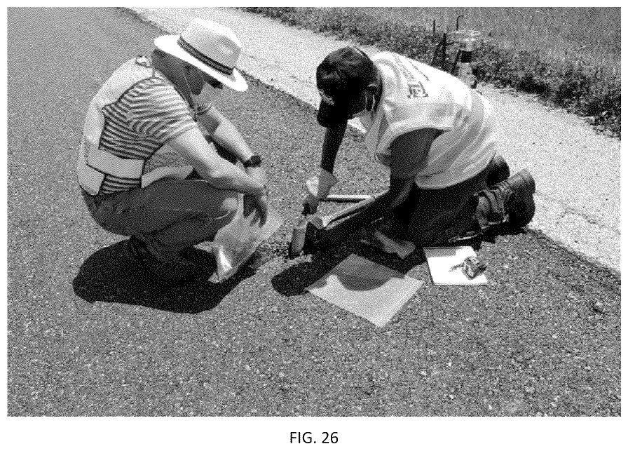

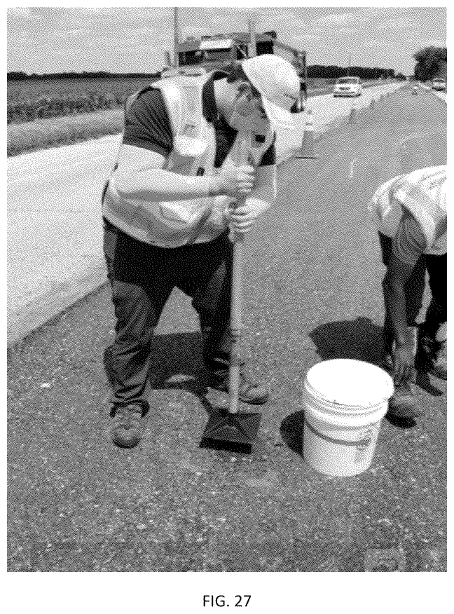

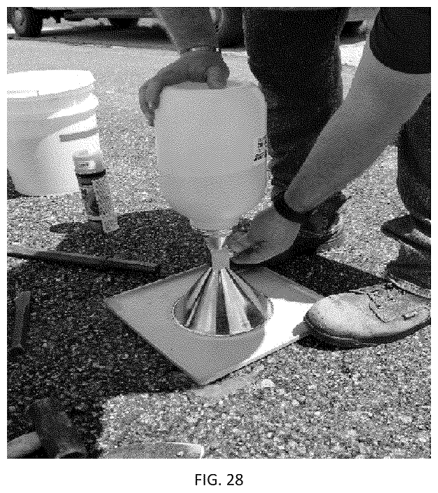

The collected samples were sealed in two layers of plastic bags to maintain moisture content (FIG. 26). Moisture content was later determined using conventional ovens at the Illinois Center for Transportation (ICT) laboratory. The holes were afterwards filled and tamped using loose mix obtained earlier from the CR conveyor (FIG. 27). Sand cone tests were also performed for density and volumetric information (FIG. 28).

From field GPR scans, the dielectric constant was calculated using the reflection amplitude (RA) method as explained before. This method of calculation is simple, common, and suitable for field applications. It is derived from Fresnel equations for EM waves' transmission and reflection. For using this method, only two inputs are needed: reflection amplitude on pavement surface (Aₚ); and reflection amplitude on top of a perfect reflecting surface (A_{c})—in this case, a copper plate is used for this purpose.

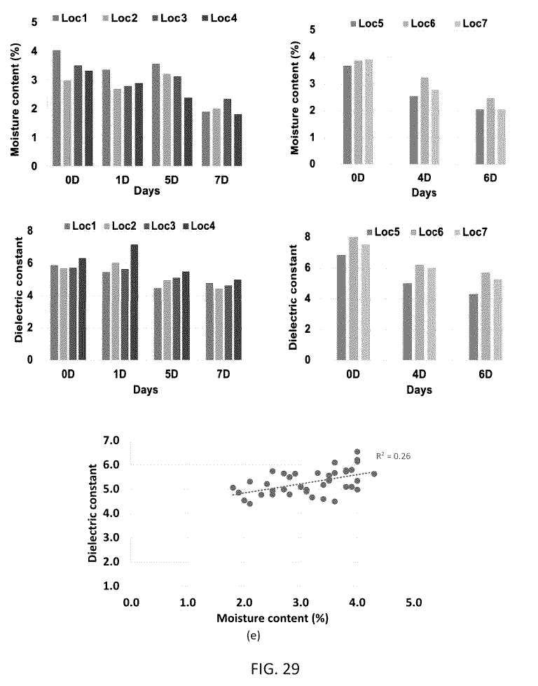

From field tests, GPR scans and corresponding measured moisture content and volumetric information were collected. FIG. 29 illustrates an example of moisture content and dielectric constant trend over a week for seven locations at the IL-61 project. Good correlation between the two parameters is observed; as moisture content decreases during curing, the dielectric constant also decreases. IL-61 data was recently used to develop a preliminary model based on the CRIM formula. The dielectric constant-moisture content relationship is not direct nor linear, because of the influence of other parameters. Hence, the electromagnetic mixing theory will be utilized for model development.

Laboratory Tests: Day-to-day variation in weather conditions, such as rain, ambient temperature, relative humidity, cloudiness, and solar radiation, could affect CR curing rate in the field; and, hence, result in spatial variability in moisture content. In addition, location-specific factors, such as shade from trees, may affect curing and evaporation processes. Consequently, laboratory testing was conducted to control some of the aforementioned field variables. In a laboratory environment, it is shade, no rain, and almost constant temperature and relative humidity. In laboratory tests, water was added at known volume to the AC slabs. Cores were taken for determining the volumetrics of the AC and moisture content was calculated.

In emulsion, water is initially bound to the asphalt binder before it breaks into free water. This change of phase is expected to cause changes in EM properties as measured by GPR. To understand the differences between bound and free water, the behaviour of conventional AC mixes with added free water was first investigated. Afterwards, emulsified CCPR mixes were tested and monitored during the curing process similar to on field testing, using GPR. The emulsions currently used in CR are usually proprietary materials-their exact composition is unknown.

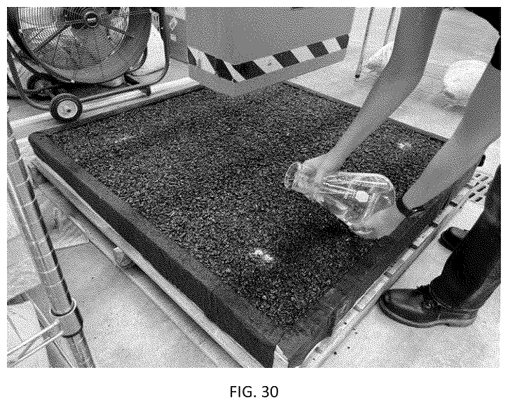

The laboratory tests included constructing and testing four slabs with similar dimensions: 4 ft×4 ft×4 in. (depth) (123 cm×123 cm×10.2 cm); the first two slabs were AC, and the other two were CCPR slabs. The slab size was selected to cover the footprint of a 2-GHz antenna placed 12 in. (30.5 cm) above the surface. This position would eliminate edge diffraction due to the wood frame. The depth of the CR slab represents common in situ CR-layer thicknesses of 3-4 in. (7.6-10.2 cm). All slabs were constructed in the same way (see slab in FIG. 30). A geomembrane was used to prevent water leakage, and a copper plate was inserted at the bottom of the slabs above the geomembrane. The copper plate was used to enable the use of the two-way travel time (TWTT) method for dielectric constant calculation (the following equation). The TWTT method allows validating the reflection amplitude method used in calculating the dielectric constant in the field. Known quantities of water were added for the AC slabs, and GPR scans were taken after each addition. More information about the laboratory slab construction and testing procedures may be found elsewhere.

$${ε}_{r} = {( \frac{c t}{2 d} )}^{2}$$

where c is EM wave propagation speed in a vacuum (3*108 m/s), t is the two-way travel time(s), and d is the layer thickness (m).

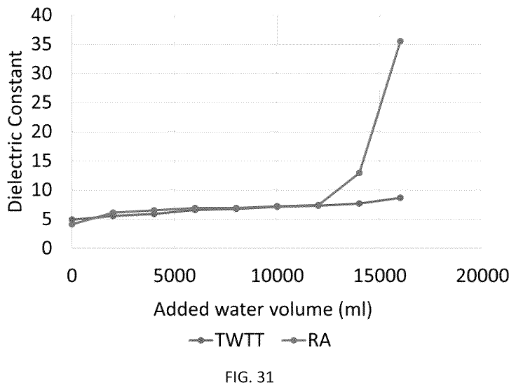

FIG. 31 illustrates the relationship between the volume of added water and the dielectric constant for the test slab. The trend is consistent-unlike in field-because various volumetric and dielectric properties of AC components are constant, while water varies. The dielectric constant was calculated using both the TWTT and the RA methods. The results from both methods matched well, except when the slab was saturated, and water accumulated on the surface. Then, the error of the reflection amplitude method increased due the masked surface reflection, which was not noticed in the field.

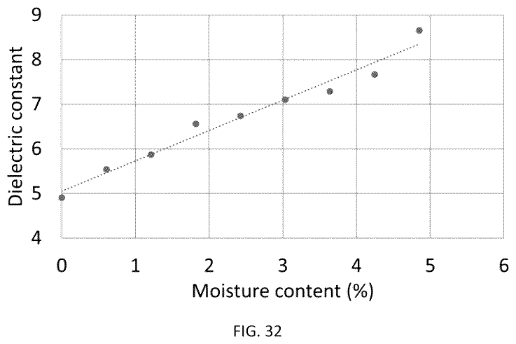

The results presented in FIG. 31 were transformed into FIG. 32 after testing and coring. Coring allowed determining the air-void percent (Pₐ), thus the total volume of voids in the slab (Vᵥ). Added volume of water (V_{w}) was transformed to gravimetric moisture content (MC) using saturation ratio (SR), Pₐ, and the maximum theoretical specific gravity (Gₘₘ). MC is commonly used by pavement engineers. The following equations explain this transformation and the table summarizes the relevant asphalt mix parameters of the second slab.

$$SR = \frac{{V}_{w}}{{V}_{v}} * 100 \%$$

$$MC = \frac{S R * {P}_{a}}{( 1 - {P}_{a} ) * {G}_{m m}}$$

| | |
|---|---|
| Percent binder (by weight) | 5.76 |
| G_{mb} (achieved) | 2.184 |
| Gₘₘ | 2.493 |
| Percent air voids (achieved) | 12.4 |
| Aggregate type | Dolomite |
| NMAS (mm) | 6.25 |
| Percent passing sieve No. 200 (by weight) | 6.75 |
| Gₛₑ | 2.730 |

Model Derivation: A large-scale testing site, having various AC material designs and layer thicknesses, was built and surveyed with GPR to correlate the bulk dielectric constant to AC dry density utilizing the Böttcher model. The testing site included six lanes of different AC mixes, comprising various aggregate types, binder grades, asphalt binder contents, densities, and AC-layer thicknesses. To calculate the AC dry density, the Böttcher EM mixing theory model was adopted for AC pavements:

$$\frac{{ε}_{eff} - {ε}_{0}}{{ε}_{eff} + u {ε}_{0} + 2 ( {ε}_{eff} - {ε}_{0} )} = {\sum }_{i = 1}^{N} {V}_{i} \frac{{ε}_{i} - {ε}_{0}}{{ε}_{i} + u {ε}_{0} + 2 ( {ε}_{eff} - {ε}_{0} )}$$

where ε_{eff} is effective or bulk dielectric constant of the mix; ε₀ is dielectric constant of background material, which is usually the most continuous material in the mix and could be considered the binder; and εᵢ is the dielectric constant of the ith inclusion for all N inclusions.

An optimized shape factor u=−0.3, which was obtained by nonlinear least square curve fitting, should be used to account for non-spherical, randomly shaped inclusions of aggregates and air in asphalt mixes. Then, AC mass-volume relationships were used to derive the Al-Qadi-Lahouar-Leng (ALL) model (shown below) for dry AC mixes, which has been proven to be accurate in predicting AC dry density (G_{mb}) on field, with an average error of only 1.1%. The model has also been verified by several global studies:

$${G}_{mb} = \frac{\frac{{ε}_{A C} - {ε}_{b}}{3 {ε}_{A C} - 2.3 {ε}_{b}} - \frac{1 - {ε}_{b}}{1 - 2.3 {ε}_{b} + 2 {ε}_{A C}}}{\frac{{ε}_{s} - {ε}_{b}}{{ε}_{s} - 2.3 {ε}_{b} + 2 {ε}_{A C}} ( \frac{1 - {P}_{d}}{{G}_{se}} ) - \frac{1 - {ε}_{b}}{1 - 2.3 {ε}_{b} + 2 {ε}_{A C}} ( \frac{1}{{G}_{mm}} )}$$

AC refers to asphalt concrete, b refers to binder, s refers to aggregates, P_{b} is percent binder (in decimal), and Gₛₑ is effective specific gravity of aggregates. In this study, the ALL model is extended to accommodate internal moisture; using the Bottcher model, it can be derived for non-dry AC case:

$$\frac{{ε}_{A C} - {ε}_{b}}{3 {ε}_{A C} - 2.3 {ε}_{b}} = {V}_{s e} \frac{{ε}_{s} - {ε}_{b}}{{ε}_{s} - 2.3 {ε}_{b} + 2 {ε}_{A C}} + {V}_{a} \frac{{ε}_{a} - {ε}_{b}}{{ε}_{a} - 2.3 {ε}_{b} + 2 {ε}_{A C}} + {V}_{w} \frac{{ε}_{w} - {ε}_{b}}{{ε}_{w} - 2.3 {ε}_{b} + 2 {ε}_{A C}}$$

where w refers to water. Substituting the following mass-volume equations, the dry density of non-dry AC may be predicted using the ACA model:

$${V}_{s e} = \frac{{G}_{m b} ( 1 - {P}_{b} )}{{G}_{s e}}$$

$${V}_{w} = w * {G}_{m b}$$

$${V}_{a} = 1 - \frac{{G}_{m b}}{{G}_{m m}} - w * {G}_{m b}$$

$${G}_{m b} = \frac{{G}_{w m b}}{( 1 + w )}$$

$${G}_{m b} = \frac{\frac{{ε}_{A C} - {ε}_{b}}{3 {ε}_{A C} - 2.3 {ε}_{b}} - \frac{1 - {ε}_{b}}{1 - 2.3 {ε}_{b} + 2 {ε}_{A C}}}{\begin{matrix}( 1 + w ) ( \frac{{ε}_{s} - {ε}_{b}}{{ε}_{s} - 2.3 {ε}_{b} + 2 {ε}_{A C}} ( \frac{1 - {P}_{b}}{{G}_{s e}} ) - \frac{1 - {ε}_{b}}{1 - 2.3 {ε}_{b} + 2 {ε}_{A C}} \\ ( \frac{1}{{G}_{m m}} ) - w ( \frac{1 - {ε}_{b}}{1 - 2.3 {ε}_{b} + 2 {ε}_{A C}} - \frac{81 - {ε}_{b}}{81 - 2.3 {ε}_{b} + 2 {ε}_{A C}} ) )\end{matrix}}$$

Conveniently, the ACA model becomes the ALL model when the moisture content (w) is set to zero. To use the ACA model to determine moisture content (e.g., cold-recycling projects), the equation has to be algebraically manipulated and the solution of the resulting quadratic equation is lengthy. This solution could be presented in a simplified way when reducing using symbols A, B, C, and D. Then, the compact solution for the moisture content be derived, which is basically one of the two roots to the quadratic equation. This results in another form of the ACA model, which could be used for moisture content prediction.

$${G}_{m b} = \frac{A}{( 1 + w ) ( B - C - w D )}$$

$$w = \frac{\begin{matrix}- ( {( {G}_{m b} ( C + D - B ) )}^{2} - 4 D {G}_{m b} \\ {( A + {G}_{m b} ( C - B ) ) )}^{1 / 2} + {G}_{m b} ( B - C - D )\end{matrix}}{2 D {G}_{m b}}$$

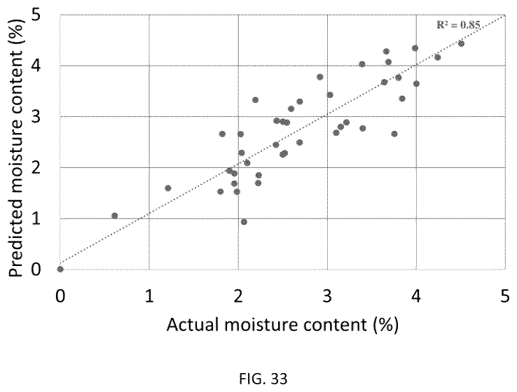

The performance of the derived ACA model in predicting dry density of wet AC and moisture content, respectively, is discussed next. The ACA model was applied to field- and laboratory-collected data, with known volumetric proportions. FIG. 33 summarizes the model's performance on AC, CIR, and CCPR materials in predicting moisture content; the R2 is 0.85, and the root mean square error (RMSE) of moisture prediction is around 0.5%. The ACA model, which is a generalizable model, also shows high accuracy in predicting dry density, as shown in the following table.

| Moisture | Sand cone | | |
|---|---|---|---|
| content | or core | Predicted | Error |
| (%) | G_{mb}* | G_{mb} | (%) |
| 1.9 | 1.956 | 1.959 | 0.2 |
| 2.5 | 1.880 | 1.916 | 1.9 |
| 3.1 | 1.906 | 1.869 | 1.9 |
| 1.8 | 1.980 | 1.955 | 1.3 |
| 2.7 | 1.954 | 2.016 | 3.1 |
| 4.0 | 2.001 | 1.964 | 1.8 |
| 3.8 | 1.929 | 1.925 | 0.2 |
| 2.1 | 2.017 | 2.016 | 0.1 |
| 2.7 | 1.833 | 1.817 | 0.9 |
| 3.4 | 1.904 | 1.968 | 3.4 |
| 5.6 | 1.775 | 1.769 | 0.3 |
| 3.4 | 1.771 | 1.724 | 2.7 |
| 2.5 | 1.796 | 1.778 | 1.0 |
| 3.7 | 1.855 | 1.891 | 2.0 |
| 2.1 | 1.902 | 1.819 | 4.4 |
| 4.0 | 1.857 | 1.891 | 1.8 |
| 5.9 | 1.884 | 1.938 | 2.9 |
| 3.8 | 1.814 | 1.727 | 4.8 |
| 4.5 | 1.797 | 1.791 | 0.4 |
| 2.4 | 1.780 | 1.781 | 0.1 |
| 2.0 | 1.787 | 1.806 | 1.0 |
| 5.2 | 1.762 | 1.732 | 1.7 |
| 3.8 | 1.727 | 1.692 | 2.0 |
| 2.5 | 1.785 | 1.808 | 1.3 |
| 2.2 | 1.714 | 1.794 | 4.7 |
| 2.0 | 1.946 | 1.939 | 0.3 |
| 2.2 | 1.941 | 1.907 | 1.8 |
| 2.9 | 1.940 | 2.029 | 4.6 |
| 2.6 | 1.873 | 1.924 | 2.7 |
| 2.0 | 1.999 | 1.955 | 2.2 |
| 2.2 | 1.955 | 1.907 | 2.4 |
| 3.2 | 1.915 | 1.882 | 1.7 |
| 2.0 | 1.883 | 1.945 | 3.3 |
| 3.2 | 1.900 | 1.865 | 1.8 |
| 0 | 2.184 | 2.185 | 0.0 |
| 0.6 | 2.184 | 2.238 | 2.5 |
| 1.2 | 2.184 | 2.231 | 2.2 |
| 1.8 | 2.184 | 2.293 | 5.0 |
| 2.4 | 2.184 | 2.247 | 2.9 |
| 3 | 2.184 | 2.235 | 2.3 |
| 3.6 | 2.184 | 2.189 | 0.2 |
| 4.2 | 2.184 | 2.173 | 0.5 |
| 4.9 | 2.184 | 2.246 | 2.8 |

*For density, sand cone tests were used in the field, whereas coring was used for the laboratory test slabs.

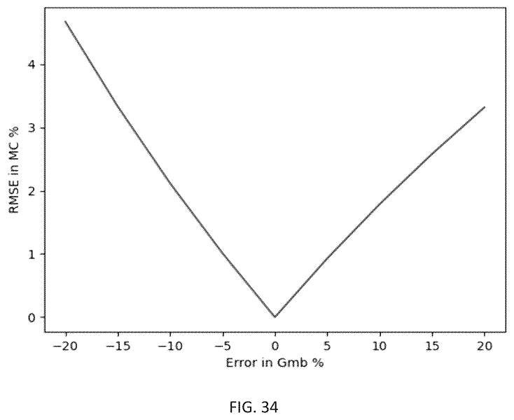

The sand cone measurements were affected by the recycled material's surface and bulk, due to the surface texture and the high air-void percent within the layer. Therefore, a sensitivity analysis was conducted to quantify the impact of this limitation on the results. Five hundred simulations were run, generating random density values corresponding to air voids of 10% to 20%, which is the typical range for cold-recycled treatments. All other parameters in the ACA model were kept constant, as would be expected in the field for a specific AC mix. As shown in FIG. 34, a 5% error in density could result in a ±1% moisture content prediction error. A small experiment on the laboratory slabs, comparing sand cone and coring measurements, showed that the error in quantifying the density is around 5%; and so field errors could be considered minor.

It is also worth mentioning that a few outliers were excluded from the analysed CIR data, the majority of which were measurements taken during the first two hours after construction. At that time, water could still be bound to emulsions. As previously mentioned, bound water has different dielectric properties than free water, which would increase prediction errors because the model is derived for free water. This effect was not noticed for AC nor CCPR.

For the cold-recycling projects, the dry density may be assumed, using laboratory mix design data, to determine moisture content; while moisture content may be assumed to predict the dry density of in-service pavements. The ACA model predicts moisture content with an RMSE less than 0.5% and the dry density with an average error of 2%. Further verification of the proposed model is needed using a larger database from different field projects.

Conclusions: Ground-penetrating radar can be effectively used for the detection and quantification of moisture content in AC pavements when accurate prediction models exist. A strong correlation between moisture content and dielectric constant was noticed for all tested materials (AC, CIR, and CCPR). A new model, Al-Qadi-Cao-Abufares (ACA), was developed to predict non-dry AC. The model developed is a modification to the Al-Qadi-Lahouar-Leng (ALL) model. The ACA model may be used in two forms, depending on the application: density prediction of non-dry AC and moisture-content prediction. The root mean square error of the ACA model in predicting moisture content is less than 0.5%; and the average error for AC dry density prediction is less than 2%, which is in line with the reported performance of the ALL model. Proper input assumptions should be made when using the model.

### Development of Aggregate Dielectric Constant Database Protocol for Asphalt Concrete Density Prediction

Contractors are paid incentives/disincentives based on achieved in-situ asphalt concrete (AC) density. Ground-penetrating radar (GPR) has been proven feasible for predicting in-situ density of AC pavements using various empirical and fundamental approaches. However, to use fundamental equations for density prediction, aggregate dielectric constant must be known beforehand. Destructive cores are usually extracted from the pavement to back-calculate the aggregate dielectric constant. This cancels out the non-destructive benefit of the GPR technology. In this study, GPR is used to quantify the dielectric constant of 10 different aggregates commonly used in AC mixes in Illinois. The sampled aggregates included Limestone, Dolomite, Traprock, Granite, and Crushed Gravel. The purpose was to initiate an aggregate dielectric constant database that would help predict AC density nondestructively and accurately. By using the aggregate database, GPR would help contractors estimate achieved density in real-time without prior calibration. This technology would save energy, time, and cost. Simulations using gprMax and a sensitivity analysis are presented to illustrate the effect of aggregate dielectric constant on AC bulk dielectric constant and, consequently, on AC density predicted by the Al-Qadi Lahouar Leng (ALL) model. A new procedure to determine the aggregate dielectric constant using the EM mixing theory is detailed. Advanced chemical tests confirmed that the aggregate dielectric constant is a function of its elemental/mineral compositions. Finally, laboratory and field data were used to validate AC density prediction using the aggregate database. The establishment of an aggregate dielectric constant database would improve the accuracy of GPR in nondestructive AC density prediction.

Ground-penetrating radar (GPR) is a subsurface nondestructive investigation technique that uses electromagnetic (EM) waves. It provides a radargram that could be used to identify subsurface features. GPR may be used to estimate material relative permittivity, which is a complex quantity describing the material's reaction to external electromagnetic (EM) fields. More recently and especially for pavement applications, dielectric profiling system (DPS) has been used. The relative permittivity of asphalt concrete (AC) is affected by its characteristics, including density, moisture content, binder content, aggregate type, size, and shape, and age. From GPR point of view, materials are distinguished based on three EM properties, namely, electric permittivity, magnetic permeability, and conductivity. Therefore, knowledge of these properties for paving materials under various conditions is necessary when interpreting GPR results. AC is considered a lossless, nonmagnetic, and nonconductive material. Consequently, the real part of the relative permittivity or the dielectric constant is the main property governing EM wave propagation in AC. Detailed explanation of GPR principles and a practical guide for application of GPR to pavements may be found elsewhere.

AC density is an important quality control/quality assurance parameter and is directly related to pavement structural capacity and performance. GPR is a feasible technique for predicting in-situ pavement AC density using various empirical and fundamental approaches. GPR is time and cost effective, has a relatively large coverage area, making it suitable for density prediction during AC layer compaction. However, some reported results for predicting AC density from GPR data could not be generalizable, due to utilizing empirical approaches. An empirical relationship is usually developed for a specific AC mix and is not valid for other AC mixes. On the other hand, fundamental approaches based on EM mixing theory could be applied to predict AC density. The dielectric mixing theory establishes a relationship between the bulk dielectric constant of AC and the dielectric constants and volumetric proportions of its components. Volumetrics of AC components could be easily obtained from the AC job mix formula. AC consists of three main components: asphalt binder, air, and aggregates. The “relative” dielectric constants of binder and air components are known to be approximately 3 and 1, respectively. Nevertheless, the aggregate dielectric constant is a function of its chemical composition and varies considerably from one type to another. Prior knowledge of the aggregate dielectric constant enables accurate AC density prediction.

Aggregate dielectric constant could be determined by extracting cores from the pavement after construction, determining their density by standard gravimetric methods, and using the cores' density to back-calculate the aggregate dielectric constant. This approach is destructive, time consuming, requires safety measures, and limits the application of this technology in real-time during AC compaction. A laboratory dielectric measurement system (LDMS) was recently proposed to measure dielectric constant of laboratory pucks prepared according to the AC mix design at different densities. A relationship is established between the measured dielectric constant and the density. This relationship can be used in the field along with DPS to estimate the in-situ AC density for the same mix. This calibration step would be omitted if aggregate dielectric constant is known, particularly considering that puck measurements are affected by edge diffraction errors.

In this study, simulations were performed using gprMax to show the major effect of aggregate dielectric constant on the AC bulk dielectric constant. Then, a sensitivity analysis was presented to show the effect of aggregate dielectric constant on the predicted density. To develop an aggregate dielectric constant database, 10 various aggregate types commonly used in Illinois (e.g., Limestone, Dolomite, Granite, Crushed Gravel, and Traprock), were tested by GPR to determine their dielectric constants using a new testing procedure. Using readily available dielectric constant values from the database allows predicting AC density nondestructively and makes real-time monitoring option feasible. The test procedure, calculations, and results are presented herein. Subsequently, advanced chemical characterization tests were conducted on the aggregate samples to understand the effect of minerology on the dielectric constant. Finally, GPR data from laboratory experiments and field projects are presented as examples for using the established aggregate database.

The determination of accurate dielectric constant is essential to predict AC density using GPR. For example, AASHTO PP98-19 sets 0.08 as a threshold for dielectric variation in air-coupled dielectric constant measurement. The dielectric constant of materials may be estimated by various methods, including coaxial-line method, resonant cavity perturbation method, free space method, and transmission/reflection method. For pavement applications, the dielectric constant could be estimated using the reflection amplitude method mentioned before. The reflection amplitude method is simple and widely applicable for pavements. Another popular method for dielectric constant estimation is the two-way travel time (TWTT) method or time of flight (TOF). However, this approach requires prior knowledge of the AC layer thickness (d) and the determination of the TWTT might not be accurate due to noise and wave attenuation. The TWTT method is recognized as providing a more representative assessment of layer properties as it considers the bulk of the layer and not only the surface reflection. There are other approaches for dielectric constant estimation. These two approaches are used in this study.

The pavement dielectric constant may be used to estimate the AC pavement density. The Al-Qadi Lahouar Leng (ALL) model is a fundamental approach based on the EM mixing theory. Other models include Bottcher, Rayleigh, Lichtenecker, and complex refractive index. The volumetric parameters can be obtained from the asphalt plant or the AC job mix formula prior to pavement construction and compaction. The AC dielectric constant is obtained from the GPR signal. For the dielectric constant of the aggregates, this study proposes obtaining it from a database. The percent of air voids can then be derived from Gₘₘ and G_{mb}. The percentage of air voids or compaction (the 100% complement) depends on the mix properties. Generally, the percentage of air voids is required to be between 2-7% for Superpave-designed AC to avoid premature failure of the pavement. Contractors are paid incentives/disincentives based on achieved in-situ density. They achieve that through rolling patterns, based on their experience or control strips/test pads. Hence, there is a need for technology that could assist contractors (and owners) to determine when desired density is achieved. Such technology would result in energy, time, and cost savings.

As seen in the ALL model, εₛ is needed as an input. Previous attempts to determine the dielectric constants of rocks were made. Geology researchers reported relationships between specific mineral contents and the dielectric constant of rocks, aiming to avoid using expensive chemical analysis and microscopic equipment to infer the chemical composition of rocks. Researchers summarized dielectric constants of some minerals tested in Japan, USA, and Korea using the parallel plate capacitor method. The values ranged between 3.4 for Sulphur and 56.0 for Samarskite (a rare radioactive mineral). Calcite (main mineral of Limestone aggregates) was reported to have dielectric constant values ranging between 7.41 and 8.6, while Dolomite (main mineral of Dolomite aggregates) ranged between 6.07 and 7.56, and Quartz ranged between 4.1 and 4.5. The range exists because of the locality of the mineral, the different proportions of elements, irregular shapes, macroscopic and microscopic defects, and crystal structure. A modified resonant cavity perturbation (RCP) method was proposed to measure the dielectric constant of aggregates and minerals. Their approach includes powdering the aggregates and mixing them with polythene then pressing them into a small plate to be tested in RCP equipment. Although this approach overcomes the heterogeneity limitations of other methods, sample preparation is time consuming, equipment has dimension and dielectric range limitations, and air is assumed to be completely extracted from the sample during pressing. In this study, we propose to determine the dielectric constant of aggregates utilizing an off-shelf GPR using a few preparation steps.

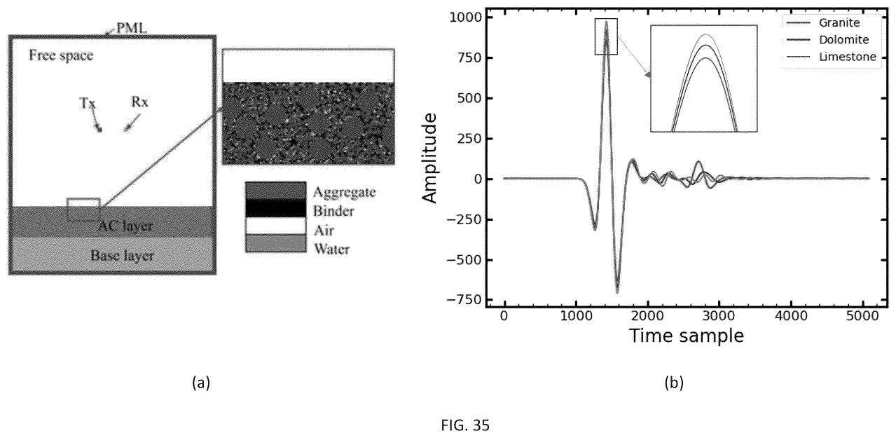

Sensitivity Analysis: Aggregate dielectric constant affects the AC bulk dielectric constant. To illustrate this, simulations of an AC layer with various aggregate dielectric constants were conducted using open-source software gprMax. GprMax solves Maxwell's equations using finite difference time domain (FDTD) technique and provides the resulting EM fields. Hence, material EM properties, such as the dielectric constant, could be calculated. The model used is a heterogeneous pavement model where different-sized aggregate, binder, and air particles were generated using random sequential adsorption (RSA) method, FIG. 35a. Tₓ is the transmitting antenna, Rₓ is the receiver antenna and PML stands for perfect matching layer, which is necessary to eliminate EM waves reflected from the boundaries of the model. The model was proposed and validated for dry and wet pavements at various moisture content levels. In this analysis, it was used to simulate dry AC at 7% air void content. Aggregate dielectric constant values of 6, 7, and 8 were used to simulate different aggregate types, which are typical values for Granite, Dolomite and Limestone, respectively. FIG. 35b shows the effect of changing the aggregate dielectric constant on the reflected GPR signal. The AC bulk dielectric constant changed from 5.12 (Granite) to 5.87 (Dolomite) to 6.06 (Limestone).

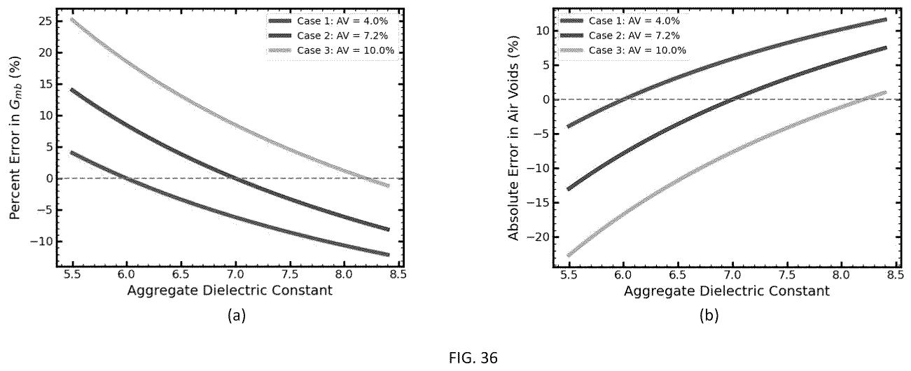

The AC density predicted by the ALL model is also sensitive to the aggregate dielectric constant. To quantitively determine this effect, three typical scenarios were studied: aggregate dielectric constants of 6, 7, and 8.2, along with three air void contents of 4.0%, 7.2%, 10.0% (typical for field AC mixes). For each scenario, aggregate dielectric constant values ranging from 5.5-8.5 were used and the predicted density (air void content) was calculated using the ALL model. FIG. 36a and FIG. 36b present the sensitivity of the predicted density and air voids to the input aggregate dielectric constant. For density, error is shown in percent difference, while for air voids, error is shown in absolute difference.

Evidently, modification in aggregate dielectric constant significantly changes predicted AC density/air voids. In the first case, with true aggregate dielectric constant of 6 and 4% air void content, when 6.5 is used as the aggregate dielectric constant, predicted air void content is 7% (3% absolute error), which is significantly different from 4%. Effects are even more pronounced for mixes with lower density (higher air void content), which aligns with previous research. This observation is reasonable, and as would be expected. Aggregates comprise around 80-85% of the AC mix by volume. Hence, slight changes in aggregate dielectric constant would significantly impact the AC bulk dielectric constant. This would impact further the calculations of AC density and other mix properties like the moisture content. Therefore, determining the dielectric properties of aggregates used in AC accurately is essential. To overcome that, establishing an aggregate database has been initiated in this work. The detailed testing methodology and analysis are presented next.

Methodology and analysis: In rocks or aggregates, the dielectric constant depends on several material characteristics, including minerology, texture, porosity, crystal structure, and water content. Aggregates from around Illinois were sampled, and test slabs were built to calculate their dielectric constants. The sampled aggregates were all of size CM16 for consistency. CM16 is an aggregate gradation band specified by IDOT which corresponds to 100% passing the 0.5 in (12.5 mm) sieve size. The aggregate's size was kept relatively small compared to the antenna wavelength to avoid EM wave diffraction from aggregate edges. In this test, a 2 GHz antenna with a wavelength of 5.9 in (150 mm) in vacuum was used.

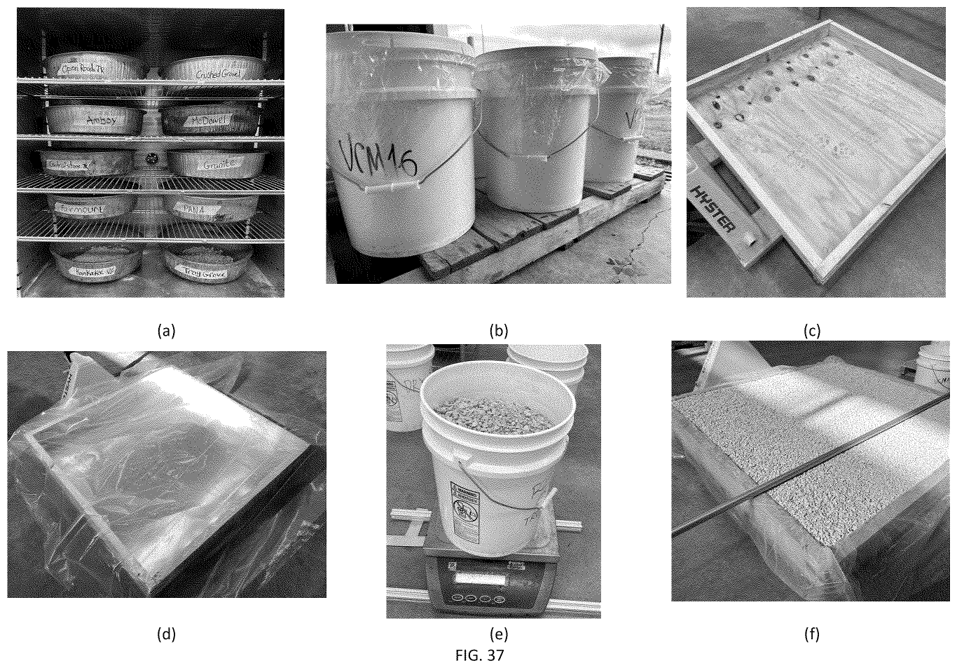

The following table presents the source quarries of the 10 aggregate products sampled from Illinois. First, the aggregates were dried overnight (24 hr) in an oven at 230° F. (110° C.) to ensure a dry condition (FIG. 37a). The dried aggregates were stored in sealed buckets until testing to avoid water adsorption from the air (FIG. 37b). This is particularly significant because moisture has a high dielectric constant of 78 at 68° F. (20° C.) compared to aggregate materials. Hence, the existence of a small amount of moisture could hinder the measured dielectric constant of the aggregates. In addition, the drying process simulates the condition of aggregates in AC mixes, which are usually heated and completely dried in the drum at the plant before being mixed and coated with asphalt binder.

| No. | Quarry Name | Product | Location |
|---|---|---|---|
| 1 | Troy Grove Quarry- | Dolomite | Utica, IL |
| | RiverStone Group, INC. | | |
| 2 | Amboy Quarry- Renner | Dolomite | Amboy, IL |
| | Quarries LTD | | |
| 3 | Kankakee Quarry- Vulcan | Dolomite | Kankakee, IL |
| | Materials | | |
| 4 | Pana Limestone | Limestone | Pana, IL |
| | Quarry Company | | |
| 5 | Fairmount Quarry- | Limestone | Fairmount, IL |
| | Heidelberg Materials | | |
| | Midwest Agg, Inc. | | |
| | (Material Service) | | |
| 6 | McDowell Quarry- | Limestone | Fairbury, IL |
| | Valley View Industries | | |
| 7 | Trap Rock & | Traprock | Ironton, MO |
| | Granite Quarries | | |
| 8 | St. Francois Quarry, | Traprock | Farmington, MO |
| | Central Stone Co. | | |
| 9 | St. Francois Quarry, | Granite | Farmington, MO |
| | Central Stone Co. | | |
| 10 | Heyworth Quarry-Rowe | Crushed | Heyworth, IL |
| | Construction Co. | Gravel | |

A wood frame of dimensions 4 ft×4 ft×4 in. (1220 mm×1220 mm×102 mm) was prepared (FIG. 37c). The wood frame dimensions allow a footprint (geometric spreading) of the antenna at 8-in.-(200-mm) height to avoid EM wave edge diffractions. A copper plate was inserted at the bottom of the frame (FIG. 37d). The copper plate allows accurate determination of TWTT for dielectric constant calculation by amplifying the reflection from the bottom of the aggregate specimen. Also, a thin plastic sheet was used to facilitate cleaning, frame reuse, and retention of fine aggregates (FIG. 37d). Each dried aggregate bucket was weighed before and after pouring the aggregates into the wood frame to accurately determine the weight of the added aggregates (FIG. 37e). The aggregates were placed gently into the wood frame to avoid any aggregate breakage or unintended compaction. The thin plastic sheet was pulled gently during the process to prevent entrapping air. Eight to nine standard five-gallon buckets were needed to completely fill the testing frame. The surface was leveled using a metallic rod to minimize wave scattering, and excess aggregates were returned to the buckets (FIG. 37f).

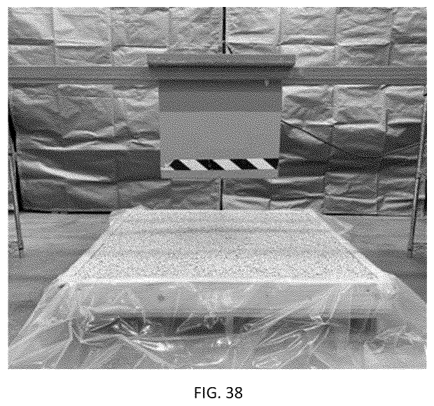

For GPR testing, FIG. 38 illustrates the test setup. The air-coupled antenna was mounted above the wood frame by supports. Calibration scans were taken on a copper plate at the same height, rate, and resolution as the tested aggregates. Due to the gentle placement of aggregates in the wood frame, the aggregates had a uniform distribution along the depth, resembling the loose unit weight (LUW) of the aggregates. This was confirmed by comparing the achieved unit weight (weight of aggregates added/volume of wood frame*1000 kg/m3) to the actual LUW tested in accordance with AASHTO T-19 specification. Readings were taken within one hour after placing the dried aggregates and scanned every five mins. Each scan duration was approximately 15 sec at 92 scans/sec rate. Most scans were taken at 1024 samples per scan to ensure high resolution. The 15-sec results were averaged into one trace or A-scan. The dielectric constant calculated by RA and TWTT matched due to homogeneity of the layer along the depth.

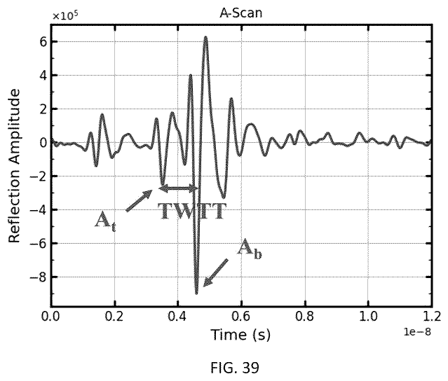

FIG. 39 shows an example GPR trace from the aggregate specimen. The x-axis shows the time window for the scan (12 ns in this experiment). The bulk dielectric constant may be calculated by TWTT method, where the TWTT is simply the time difference between the top reflection (Aₜ) of the specimen and the bottom reflection (A_{b}), which is amplified by the embedded copper plate. In this case, the bulk dielectric constant is not equal to the aggregate dielectric constant because of the entrapped air particles between the aggregates. Having a definite weight of the added aggregates and knowing their specific gravity (provided by the Illinois Department of Transportation), the aggregate volume could be calculated using the following equation. The aggregates comprised 50-60% of the wood frame volume for all the tested aggregates, aligning well with the LUW of the aggregates. The volume of air could be approximated by subtracting the volume of aggregates from the total internal volume of the wood frame. Then, the Bottcher mixing theory model could be used to solve for the aggregate dielectric constant.

$${V}_{a g g} = \frac{{W}_{a g g}}{{G}_{s b} * 1000}$$

where V_{agg} is volume of aggregates (m3), W_{agg} is total weight of aggregates added to the slab (g) and G_{sb} is bulk specific gravity of aggregates provided by IDOT.

The Bottcher mixing theory model is the base model for the ALL density prediction model; hence, it was chosen for consistency. In the test case, the only mix components are air and aggregates, and can be written as the following equation using a shape factor of u=−0.3 to account for irregular-shaped aggregate inclusions:

$$\frac{{ε}_{b u l k} - 1}{3 {ε}_{b u l k} - 2.3} = {V}_{a g g} \frac{{ε}_{a g g} - 1}{{ε}_{a g g} + 2 {ε}_{b u l k} - 2.3}$$

Data processing included simple shift removal, signal instability and height corrections, and Fourier interpolation for low-resolution data points. The following table shows the calculated aggregate dielectric constant values.

| Aggregate | | Specific Gravity of | Dielectric | | Coefficient of |
|---|---|---|---|---|---|
| Type | Quarry Location | Aggregates (G_{sb}) | Constant (ε) | Average | Variation (CoV) |
| Dolomite | Troy Grove, IL | 2.632 | 7.40-7.57 | 7.35 | 0.03 |
| Dolomite | Amboy, IL | 2.618 | 7.02-7.20 | | |
| Dolomite | Kankakee, IL | 2.611 | 7.37-7.56 | | |
| Limestone | Pana, IL | 2.600 | 8.10 | 8.06 | 0.04 |
| Limestone | Fairmount, IL | 2.590 | 8.25-8.47 | | |
| Limestone | McDowell, IL | 2.582 | 7.64-7.82 | | |
| Trap Rock | Ironton, MO | 2.635 | 6.60 | 6.25 | 0.07 |
| Trap Rock | Farmington, MO | 2.593 | 5.80-5.99 | | |
| Granite | Farmington, MO | 2.587 | 5.96-6.14 | 6.05 | 0.02 |
| Crushed | Heyworth, IL | 2.605 | 6.50-6.67 | 6.59 | 0.02 |
| Gravel | | | | | |

From the previous table, there is a small range of dielectric constant values for some of the tested aggregates. This variation could be attributed to surface wave scattering due to the rough aggregate surface, or prolonged testing, which may have caused the aggregates to absorb some water from the air. For the same type of aggregate (e.g., Limestone), the dielectric constant differed per source due to minerology, crystal structure, and geometric shape. Average values in Table 2 could be directly used in AC density prediction models. The obtained values agree with reported values in the literature. For a deeper understanding of the minerology effects on dielectric constant, samples from the 10 aggregates were chemically characterized at the Materials Research Laboratory at the University of Illinois Urbana-Champaign.

Advanced chemical analysis: X-ray diffraction (XRD) and X-ray fluorescence (XRF) tests were performed for compound (mineral) and elemental characterization, respectively. Two samples from each aggregate were tested to check variation within aggregate type. XRD allows for classification of the aggregate types using main minerals detected based on crystal structure. For example, for the three Dolomite aggregates, the Dolomite mineral (MgCa(CO₃)₂) was dominant (>90%), and for Limestone aggregates, Calcite (CaCO₃) was dominant (>85%). The two different Traprock aggregates showed significant difference in their mineral composition: the one from Ironton mainly had 68% Sanidine (KAlSi₃O₈) and 32% Silica (SiO₂), while the one from Farmington had 45% Silica and 44% Albite (NaAlSi₃O₈). This might explain the difference in their dielectric constants in the previous table. Traprock is a broad category of dark non-granitic igneous rocks. Sampled Granite had about 48% Silica (SiO₂), 31% Albite (NaAlSi₃O₈) and 21% Sanidine (KAlSi₃O₈), which is similar to the composition of the Traprock obtained from the same quarry; hence, similar dielectric results. For the Crushed Gravel, one sample was classified as Limestone while the other was classified as Dolomite, which suggests that these aggregates are highly variable in structure and further analysis such as elemental characterization should be done.

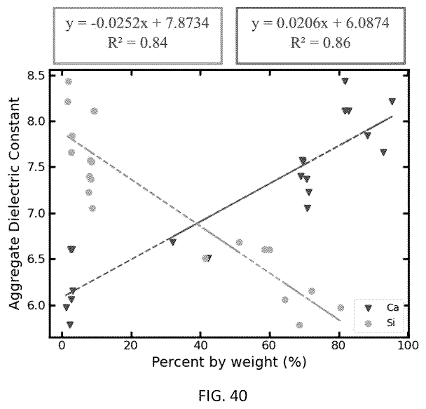

XRF analysis provided the elemental composition, which can offer insights into impurities and elemental effects on the dielectric properties. For example, FIG. 40 shows the effect of Calcium (Ca) and Silicon (Si) contents on the aggregate dielectric constant for the 20 samples. As expected, Dolomite and Limestone aggregates with high Ca content exhibited relatively high dielectric constants. In contrast, aggregates with more Si content, such as Granite and Traprock, had lower dielectric constants. This is due to the higher dielectric constant of the Ca element compared to that of Si. Generally, aggregates with similar elemental composition had similar dielectric constant results. Also, the Limestone samples with the highest dielectric constant from Fairmount had around 8% Iron (Fe), which would significantly increase the dielectric constant. Granite had the highest percentage of Si (around 78%). This explains its relatively low dielectric constant compared to other aggregate types. The two Crushed Gravel samples have similar elemental composition; however, they have different crystal or mineral structures. This resulted in XRD classification of one as Dolomite while the other as Limestone. Nevertheless, the Crushed Gravel samples have a high Si content (higher than Ca or Mg commonly seen in Limestone and Dolomite). Hence, the calculated dielectric constant is lower than that of Limestone or Dolomite. The Si content was not detected in the Crushed Gravel XRD results. This could be due to overlapping peaks in the XRD spectrum or high amorphous content. The following table briefly presents the XRF results by averaging the results of two samples for each aggregate. Only the top three elements by weight percentage are presented herein, whereas each aggregate type has at least 11 elements detected.

| Aggregate | Quarry | Element | Element | Element |
|---|---|---|---|---|
| type | Location | #1 | #2 | #3 |
| Dolomite | Troy Grove, IL | Ca (69.2%) | Mg (13.1%) | Si (8.0%) |
| Dolomite | Amboy, IL | Ca (71.0%) | Mg (16.3%) | Si (8.3%) |
| Dolomite | Kankakee, IL | Ca (70.2%) | Mg (15.1%) | Si (8.5%) |
| Limestone | Pana, IL | Ca (82.2%) | Si (9.3%) | Al (3.4%) |
| Limestone | Fairmount, IL | Ca (88.6%) | Fe (7.8%) | Si (1.7%) |
| Limestone | McDowell, IL | Ca (90.5%) | Fe (3.0%) | Si (2.9%) |
| Trap Rock | Ironton, MO | Si (59.3%) | K (14.9%) | Al (13.0%) |
| Trap Rock | Farmington, MO | Si (66.5%) | Al (12.7%) | K (11.6%) |
| Granite | Farmington, MO | Si (76.3%) | K (8.5%) | Al (7.7%) |
| Crushed | Heyworth, IL | Si (46.3%) | Ca (37.1%) | Mg (5.4%) |
| Gravel | | | | |

This simple chemical analysis shows the direct connection and correlation between aggregate minerology and dielectric properties. The elemental composition was found to be more influential than mineral composition, especially when impurities that alter dielectric constant exist (e.g., Iron). In general, chemical composition could assist in predicting the dielectric properties of aggregates.

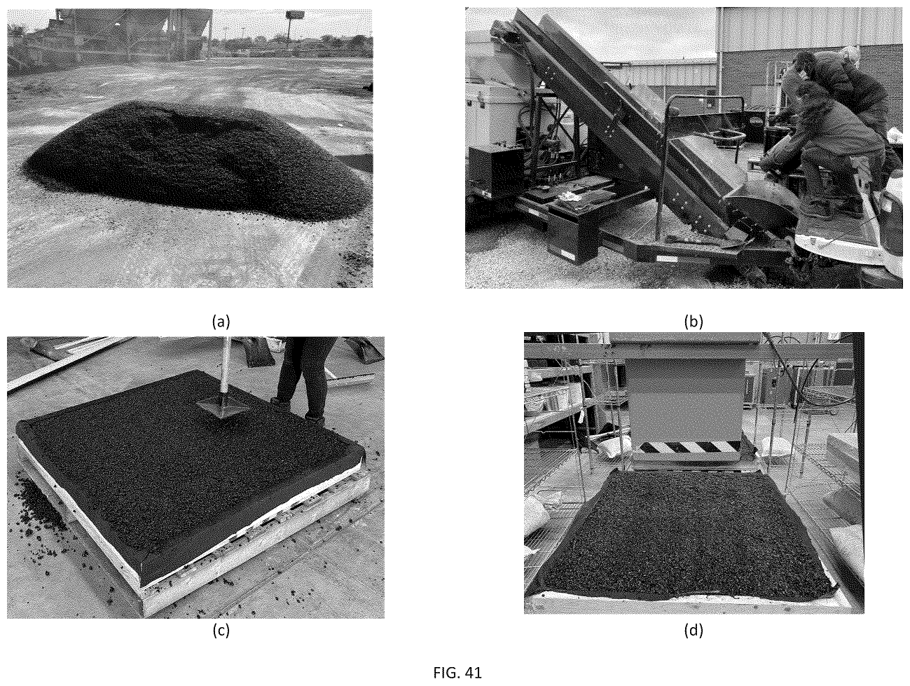

Validation and discussion: For validation, a highway mix and an FAA-approved airport AC mix were tested using GPR. The density was predicted by the ALL model using aggregate dielectric constants directly from the established database. The airport AC mix was sampled from Sandeno East, IL, FIG. 41a; reheated and mixed in a mobile mixer, FIG. 41b; placed and compacted into a 4 in (100 mm) layer using hand tamping, FIG. 41c. FIG. 41d shows the resulting AC specimen and the test setup. The bulk dielectric constant (ε_{AC}) was calculated using TWTT method. Three cores were extracted from the specimen after GPR measurements. Corelok method (AASHTO T-331) was used to determine the ground truth bulk specific gravities (G_{mb}) from the cores. The average ground truth specific gravity was calculated at 2.093, which corresponded to 16.2% air voids. Maximum theorical specific gravity (Gₘₘ), binder content (P_{b}), and effective specific gravity of aggregates (Gₛₑ) were obtained from the mix design sheet. Over 80% of the aggregate blend is Limestone from Thornton quarry, IL. Therefore, according to the database, an average aggregate dielectric constant value of 8.06 was used. The following table presents the inputs to the ALL model along with the predicted AC density. The predicted G_{mb} is 2.077, which corresponds to 16.8% air voids (AV). The results agree with the ground truth values. The slight difference could be attributed to the minerology of Thornton quarry aggregates, which might differ slightly from the Limestone entries in the database, as well as the effect of the other 20% aggregate in the blend.

| ALL Model Inputs | | | | | Output | |
|---|---|---|---|---|---|---|
| P_{b} (%) | Gₘₘ | Gₛₑ | εₛ | ε_{AC} | G_{mb} | AV (%) |
| 6.1 | 2.497 | 2.750 | 8.06 | 4.85 | 2.077 | 16.8 |

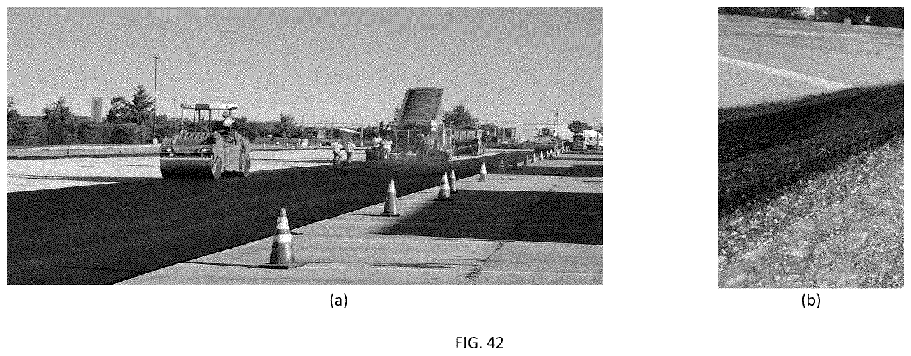

A Home Depot parking lot in Joliet, IL was used for field validation in September 2022. The project was paved by Gallagher Asphalt, which included a 12 in (300 mm) reconstruction in three AC lifts: 6 in (150 mm) binder, 4 in (100 mm) intermediate, and 2 in (50 mm) wearing surface. FIG. 42 shows the construction site and pavement cross-section.

GPR data were collected before, during, and after construction of the first lift. The length of one lane segment was approximately 780 ft (238 m). RA method was used to estimate the dielectric constant for several locations continuously along the lanes. Later, the nuclear gauge operators reported air voids of 7-7.5% for sporadic locations for the first lift on the first lane. This project mix used 60% aggregates from Thornton, IL and 20% from Manteno, IL; both quarries produce mainly Limestone. The following table presents the AC density and air void content calculated using the ALL model for the last continuous scan on the first lane. The estimated dielectric constant was averaged over the lane for the comparison. The predicted G_{mb} is 2.341 which corresponds to 7.9% air voids.

| ALL Model Inputs | | | | | Output | |
|---|---|---|---|---|---|---|
| P_{b} (%) | Gₘₘ | Gₛₑ | εₛ | ε_{AC} | G_{mb} | AV (%) |
| 5.1 | 2.542 | 2.760 | 8.06 | 6.25 | 2.341 | 7.9 |

The results are close to the values produced by the nuclear gauge. The difference in results in this case could be attributed to the following: 1) averaging the dielectric constant across the lane; 2) the use of 20% fractionated reclaimed asphalt pavement (FRAP), for which the aggregate type is unknown and has aged binder; and 3) the accuracy of nuclear gauge is unknown.

The aforementioned cases demonstrate the potential of predicting accurate AC density using GPR. However, it is evident that aggregate type and mineralogy have significant impact on AC density prediction. Hence, creating an aggregate dielectric constant database is essential to ensure accurate AC prediction using GPR.

Conclusions: 1) A new protocol is proposed to estimate the dielectric constant of aggregates used in AC mixes using GPR and EM mixing theory 2) The sensitivity of AC bulk dielectric constant and predicted density to aggregate dielectric constant highlights the importance of establishing an aggregate database for real-time nondestructive AC density prediction in asphalt pavements 3) Advanced XRD and XRF analyses confirm that aggregate dielectric constant is influenced by aggregate mineralogy and chemical composition 4) A 10-aggregate database was established for the State of Illinois. The aggregate database may be extended to other states to include all aggregates used in the pavement industry. The aggregate database approach was validated against laboratory and field tests.

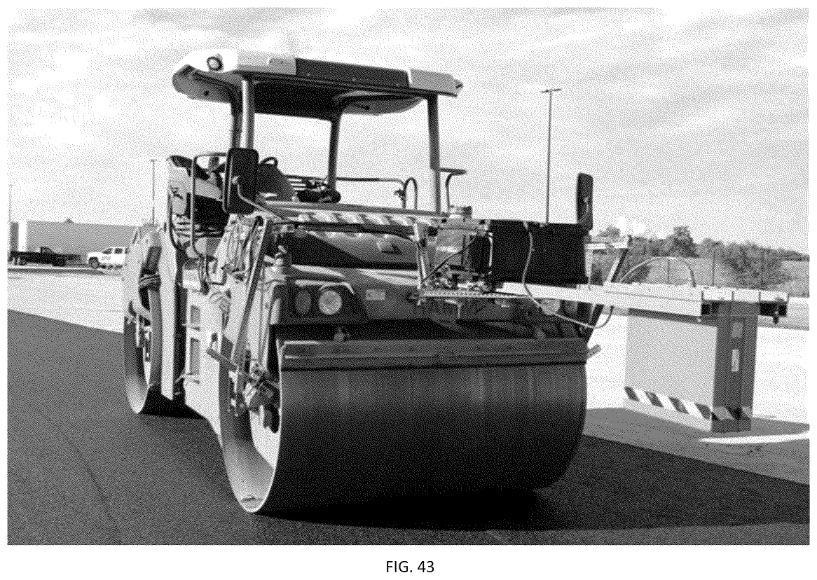

In various embodiments, FIG. 43 shows an image of the mount on a field project.

In various embodiments in the present disclosure, a module may refer to a software module, a hardware module, or a combination thereof. A software module may include a computer program or part of the computer program that has a predefined function and works together with other related parts to achieve a predefined goal, such as those functions described in this disclosure. A hardware module may be implemented using processing circuitry and/or memory configured to perform the functions described in this disclosure. Each module can be implemented using one or more processors (or processors and memory). Likewise, a processor (or processors and memory) can be used to implement one or more modules. Moreover, each module can be part of an overall module that includes the functionalities of the module. The description here also applies to the term module and other equivalent terms.

In the present disclosure, the term “processor” means one processor that performs the defined functions, steps, or operations or a plurality of processors that collectively perform defined functions, steps, or operations, such that the execution of the individual defined functions may be divided amongst such plurality of processors.

The methods, devices, processing, and logic described above may be implemented in many different ways and in many different combinations of hardware and software. For example, all or parts of the implementations may be circuitry that includes an instruction processor, such as a Central Processing Unit (CPU), microcontroller, or a microprocessor; an Application Specific Integrated Circuit (ASIC), Programmable Logic Device (PLD), or Field Programmable Gate Array (FPGA); or circuitry that includes discrete logic or other circuit components, including analog circuit components, digital circuit components or both; or any combination thereof. The circuitry may include discrete interconnected hardware components and/or may be combined on a single integrated circuit die, distributed among multiple integrated circuit dies, or implemented in a Multiple Chip Module (MCM) of multiple integrated circuit dies in a common package, as examples.

The circuitry may further include or access instructions for execution by the circuitry. The instructions may be embodied as a signal and/or data stream and/or may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other magnetic or optical disk; or in or on another machine-readable medium. A product, such as a computer program product, may particularly include a storage medium and instructions stored in or on the medium, and the instructions when executed by the circuitry in a device may cause the device to implement any of the processing described above or illustrated in the drawings.

The implementations may be distributed as circuitry, e.g., hardware, and/or a combination of hardware and software among multiple system components, such as among multiple processors and memories, optionally including multiple distributed processing systems. Parameters, databases, and other data structures may be separately stored and managed, may be incorporated into a single memory or database, may be logically and physically organized in many different ways, and may be implemented in many different ways, including as data structures such as linked lists, hash tables, arrays, records, objects, or implicit storage mechanisms. Programs may be parts (e.g., subroutines) of a single program, separate programs, distributed across several memories and processors, or implemented in many different ways, such as in a library, such as a shared library (e.g., a Dynamic Link Library (DLL)). The DLL, for example, may store instructions that perform any of the processing described above or illustrated in the drawings, when executed by the circuitry.

While the particular disclosure has been described with reference to illustrative embodiments, this description is not meant to be limiting. Various modifications of the illustrative embodiments and additional embodiments of the disclosure will be apparent to one of ordinary skill in the art from this description. Those skilled in the art will readily recognize that these and various other modifications can be made to the exemplary embodiments, illustrated and described herein, without departing from the spirit and scope of the present disclosure. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

What is claimed is:

1. A system for providing compaction status of a continuous asphalt concrete in real-time, the system comprising:
a controller comprising a memory storing instructions, and the controller comprising a processor in communication with the memory, wherein, when the processor executes the instructions, the processor is configured to cause the controller to perform:
Using raw data from a ground penetrating radar sensing an asphalt concrete at a location;
processing the raw data to obtain processed data;
determining an asphalt density of the asphalt concrete at the location based on the processed data according to an asphalt density prediction model;
determining a compaction status of the asphalt concrete at the location based on the asphalt density and a target compaction threshold; and
outputting the compaction status of the asphalt concrete at the location to an operator for determining whether to continue or stop compacting the asphalt concrete at the location.

2. The system according to claim 1, wherein, when the processor executes the instructions, the processor is configured to cause the controller to perform:
while the ground penetrating radar is moving along the asphalt concrete, repeating steps from using collected raw data to the outputting the compaction status with respect to the asphalt concrete continuously at a plurality of locations.

3. The system according to claim 1, further comprising:
a mounting structure configured to mount the controller and the ground penetrating radar to a compaction roller, wherein the mounting structure comprises:
a plurality of rigid bars configured to fix to lifting points of the compaction roller;
a truss structure fixed to the plurality of rigid bars and configured to receive the controller; and
a plurality of beams, wherein, for each beam of the plurality of beams, a proximal end of the beam is configured to fix to the truss structure and a distal end of the beam is configured to mount the ground penetrating radar.

4. The system according to claim 3, wherein:
the plurality of rigid bars comprise:
a telescopic rigid bar with an adjustable length configured to fit a width of the compaction roller, and
one or more rigid bars with adjustable lengths configured to fix to the lifting points of the compaction roller; and
the plurality of beams comprise non-metallic material.

5. A method for providing compaction status of a continuous asphalt concrete in real-time performed by an electronic device, the method comprising:
using obtained raw data from a ground penetrating radar sensing asphalt concrete at a location;
processing the raw data to obtain processed data;
determining an asphalt density of the asphalt concrete at the location based on the processed data according to an asphalt density prediction model;
determining a compaction status of the asphalt concrete at the location based on the asphalt density and a target compaction threshold; and
outputting the compaction status of the asphalt concrete at the location to an operator for determining whether to continue or stop compacting the asphalt concrete at the location.

6. The method according to claim 5, further comprising:
while the ground penetrating radar is moving along the asphalt concrete during compaction, repeating steps from the obtaining raw data to the outputting the compaction status with respect to the asphalt concrete continuously at a plurality of locations.

7. The method according to claim 5, wherein, the determining the compaction status of the asphalt concrete at the location based on the asphalt density and the target compaction threshold comprises:
in response to the asphalt density being smaller than the target compaction threshold, determining the compaction status of the asphalt concrete at the location as under-compaction with a color code corresponding to the asphalt density;
in response to the asphalt density being equal to the target compaction threshold, determining the compaction status of the asphalt concrete at the location as at-target-compaction with a color code corresponding to the asphalt density; and
in response to the asphalt density being larger than the target compaction threshold, determining the compaction status of the asphalt concrete at the location as over-compaction with a color code corresponding to the asphalt density.

8. The method according to claim 5, wherein:
the target compaction threshold corresponds to percent compaction specified by designer/owner.

9. The method according to claim 5, wherein, the outputting the compaction status of the asphalt concrete at the location to an operator for determining whether to continue or stop compacting the asphalt concrete at the location comprises:
generating a heatmap based on color codes for the asphalt concrete at a plurality of locations; and
outputting the heatmap to an operator for determining whether to continue or stop compacting the asphalt concrete at the location.

10. The method according to claim 5, wherein:
the compaction status of the asphalt concrete at the location being under-compaction is configured for the operator to continue compacting the asphalt concrete at that location; or
the compaction status of the asphalt concrete at the location being at target compaction is configured for the operator to stop compacting the asphalt concrete at that the location.

11. The method according to claim 5, further comprising:
obtaining geo-location data of the asphalt concrete at the location from a global positioning system; and
associating the geo-location data and the asphalt density of the asphalt concrete at the location.

12. The method according to claim 5, wherein:
the processing the raw data comprises at least one of the following: pre-processing the raw data, performing signal instability correction; performing height correction; performing thin layer processing; performing surface moisture removal; and/or performing wavelet smoothing.

13. The method according to claim 5, wherein:
the ground penetrating radar is configured to mount to a compaction roller by a mounting structure, wherein the mounting structure comprises:
a plurality of rigid bars configured to fix to lifting points of the compaction roller;
a truss structure fixed to the plurality of rigid bars and configured to receive the electronic device; and
a plurality of beams, wherein, for each beam of the plurality of beams, a proximal end of the beam is configured to fix to the truss structure and a distal end of the beam is configured to mount the ground penetrating radar.

14. A method for providing moisture content of a continuous asphalt concrete in real-time performed by an electronic device, the method comprising:
obtaining raw data from a ground penetrating radar sensing an asphalt concrete at a location;
processing the raw data to obtain processed data;
determining a moisture content of the asphalt concrete at the location based on the processed data according to a moisture content prediction model;
determining a moisture status of the asphalt concrete at the location based on the moisture content and a target moisture threshold; and
outputting the moisture status of the asphalt concrete at the location to an operator for determining whether to close or open the asphalt concrete for traffic.

15. The method according to claim 14, further comprising:
while the ground penetrating radar is moving along the asphalt concrete, repeating from the obtaining raw data to the outputting the moisture status with respect to the asphalt concrete continuously at a plurality of locations.

16. The method according to claim 14, further comprising:
calibrating for the existing material prior to cold recycling for moisture content prediction model.

17. The method according to claim 14, wherein, the determining the moisture status of the cold recycled asphalt concrete at the location based on the moisture content and the target moisture threshold comprises:
in response to the moisture content being smaller than or equal to the target moisture threshold, determining the moisture status of the cold recycled asphalt concrete at the location as dry; and
in response to the moisture content being larger than the target moisture threshold, determining the moisture status of the asphalt concrete at the location as wet.

18. The method according to claim 14, wherein:
the target moisture threshold comprises a percent moisture set by the designer/owner.

19. The method according to claim 14, wherein:
the moisture status of the cold recycled asphalt concrete at the location being dry is configured for the operator to open the asphalt concrete for traffic; and
the moisture status of the cold recycled asphalt concrete at the location being wet is configured for the operator to keep closing the cold recycling asphalt concrete for traffic.

20. The method according to claim 14, wherein:
the ground penetrating radar is configured to mount on a moving vehicle, wherein the mounting structure comprises:
a plurality of rigid bars configured to fix to the vehicle;
a truss structure fixed to the plurality of rigid bars; and
a plurality of beams, wherein, for each beam of the plurality of beams, a proximal end of the beam is configured to fix to the truss structure and a distal end of the beam is configured to mount the ground penetrating radar.
